SEQUENCE LISTING (77 sequences)

SEQ ID NO: 1 - protein, 24 aa, synthetic construct
  REGION 1..24: note = programmed membrane proteins proMPs
PLSFLLGILAALVGFIIGFLAALI

SEQ ID NO: 2 - protein, 24 aa, synthetic construct
  REGION 1..24: note = programmed membrane proteins proMPs
PLLFILVAILGGLFGAIVAFLLAL

SEQ ID NO: 3 - protein, 24 aa, synthetic construct
  REGION 1..24: note = programmed membrane proteins proMPs
PILFAILAAFIGAFIAALFVLVLA

SEQ ID NO: 4 - protein, 24 aa, synthetic construct
  REGION 1..24: note = programmed membrane proteins proMPs
PFGALLAIIAFVVGMLFSAFVLLI

SEQ ID NO: 5 - protein, 24 aa, synthetic construct
  REGION 1..24: note = programmed membrane proteins proMPs
PFSLFLGVIAGIIAAFIVLFLALL

SEQ ID NO: 6 - protein, 24 aa, synthetic construct
  REGION 1..24: note = programmed membrane proteins proMPs
PFLSLVGALIGAFIAFILALFILV

SEQ ID NO: 7 - protein, 24 aa, synthetic construct
  REGION 1..24: note = programmed membrane proteins proMPs
PILITLAMLTGAVIGAISSFLLVY

SEQ ID NO: 8 - protein, 24 aa, synthetic construct
  REGION 1..24: note = programmed membrane proteins proMPs
PAFYIILAILTSFIAYLVGLLVSF

SEQ ID NO: 9 - protein, 24 aa, synthetic construct
  REGION 1..24: note = programmed membrane proteins proMPs
PIYVILAILAAVFTWFIVLITSLS

SEQ ID NO: 10 - protein, 24 aa, synthetic construct
  REGION 1..24: note = programmed membrane proteins proMPs
PTVTSAILGVSFGTFISLVALWLA

SEQ ID NO: 11 - protein, 24 aa, synthetic construct
  REGION 1..24: note = programmed membrane proteins proMPs
PVIAILTFIVLTAISGALLAVWFS

SEQ ID NO: 12 - protein, 24 aa, synthetic construct
  REGION 1..24: note = programmed membrane proteins proMPs
PIVLLLSLLASVFGAFITFIWAYL

SEQ ID NO: 13 - protein, 24 aa, synthetic construct
  REGION 1..24: note = programmed membrane proteins proMPs
PLTVALILGIFLGTFIAFWVVYLL

SEQ ID NO: 14 - protein, 24 aa, synthetic construct
  REGION 1..24: note = programmed membrane proteins proMPs
TALLVAFVAYYTALIALIFAILAT

SEQ ID NO: 15 - DNA, 72 bp, synthetic construct
  misc_feature 1..72: note = programmed membrane proteins proMPs
  source 1..72: mol_type = other DNA
cctttatctttcctcttagggatactagctgcgctggtggggttcatcattggctttttagcggccttgatt

SEQ ID NO: 16 - DNA, 72 bp, synthetic construct
  misc_feature 1..72: note = programmed membrane proteins proMPs
  source 1..72: mol_type = other DNA
cctttgttatttattctcgtcgcaatacttggaggcttatttggggcgattgttgcattccttttggcgtta

SEQ ID NO: 17 - DNA, 72 bp, synthetic construct
  misc_feature 1..72: note = programmed membrane proteins proMPs
  source 1..72: mol_type = other DNA
ccgatcctgttcgcaatactggcggctttcatcggggcatttatagctgccctgttcgtgctagtattggca

SEQ ID NO: 18 - DNA, 72 bp, synthetic construct
  misc_feature 1..72: note = programmed membrane proteins proMPs
  source 1..72: mol_type = other DNA
ccctttggagctttactagcaatcatagcattcgtcgtaggaatgttattctcagcattcgttttactcatc

SEQ ID NO: 19 - DNA, 72 bp, synthetic construct
  misc_feature 1..72: note = programmed membrane proteins proMPs
  source 1..72: mol_type = other DNA
ccctttagcttgtttttgggcgttatagccggcattattgctgcattcatcgttttattcctggcattacta

SEQ ID NO: 20 - DNA, 72 bp, synthetic construct
  misc_feature 1..72: note = programmed membrane proteins proMPs
  source 1..72: mol_type = other DNA
ccttttttatcgcttgttggtgcgctaatcggggctttcatagcatttatcttggctttgttcattttggtt

SEQ ID NO: 21 - DNA, 72 bp, synthetic construct
  misc_feature 1..72: note = programmed membrane proteins proMPs
  source 1..72: mol_type = other DNA
ccgattctgatcactttggcaatgcttacgggagcagtgattggggcgatctcgtcttttctcctagtgtat

SEQ ID NO: 22 - DNA, 72 bp, synthetic construct
  misc_feature 1..72: note = programmed membrane proteins proMPs
  source 1..72: mol_type = other DNA
ccagccttttatattatattggcaattctcacctcgttcatagcctatttggtgggtctactcgtgtctttt

SEQ ID NO: 23 - DNA, 72 bp, synthetic construct
  misc_feature 1..72: note = programmed membrane proteins proMPs
  source 1..72: mol_type = other DNA
cctatttacgttatactagccatcttggccgcggtattcacttggttcatagtccttataactagcctgagt

SEQ ID NO: 24 - DNA, 72 bp, synthetic construct
  misc_feature 1..72: note = programmed membrane proteins proMPs
  source 1..72: mol_type = other DNA
cctacggttacgagtgcgattcttggcgtgtcattcggtacctttattagcctcgtagctctgtggcttgca

SEQ ID NO: 25 - DNA, 72 bp, synthetic construct
  misc_feature 1..72: note = programmed membrane proteins proMPs
  source 1..72: mol_type = other DNA
ccagtgattgcaatcttaacttttatagtcctcactgcgatttcgggagcgctgctcgctgtttggttctcc

SEQ ID NO: 26 - DNA, 72 bp, synthetic construct
  misc_feature 1..72: note = programmed membrane proteins proMPs
  source 1..72: mol_type = other DNA
cccatcgtcttgctcctcagtctactcgccagtgtatttggggcgttcatcacatttatttgggcttacttg

SEQ ID NO: 27 - DNA, 72 bp, synthetic construct
  misc_feature 1..72: note = programmed membrane proteins proMPs
  source 1..72: mol_type = other DNA
ctggtgctgattctgctgacctttgtgctgtttgtgtttattctgtattgggtgattacctggtatctgatt

SEQ ID NO: 28 - DNA, 72 bp, synthetic construct
  misc_feature 1..72: note = programmed membrane proteins proMPs
  source 1..72: mol_type = other DNA
ccgctgaccgtggcgctgattctgggcatcttcctgggcacctttattgcgttttgggtggtgtatctgctg

SEQ ID NO: 29 - DNA, 72 bp, synthetic construct
  misc_feature 1..72: note = programmed membrane proteins proMPs
  source 1..72: mol_type = other DNA
accgcgctgctggtggcgtttgtggcgtattataccgcgctgattgcgctgatttttgcgattctggcgacc

SEQ ID NO: 30 - DNA, 71 bp, synthetic construct
  misc_feature 1..71: note = monomeric C-terminal portion of human L-selectin (CLS)
  source 1..71: mol_type = other DNA
ccgctgttcatcccggttgcagttatggttaccgcttttagtggattggggtttatcatctggctggctac

SEQ ID NO: 31 - DNA, 68 bp, synthetic construct
  misc_feature 1..68: note = ErbB2 (erb-b2 receptor tyrosine kinase 2)
  source 1..68: mol_type = other DNA
tctatcatctctgcggtggttggcattctgctggtcgtggtcttgggcgtggtctttggcatcctgat

SEQ ID NO: 32 - DNA, 57 bp, synthetic construct
  misc_feature 1..57: note = QSOX2 (quiescin sulfhydryl oxidase 2)
  source 1..57: mol_type = other DNA
agcctatgcgttgttttatacgtggcatctagtttatttatggtcatgtacttcttc

SEQ ID NO: 33 - skipped

SEQ ID NO: 34 - protein, 4 aa, synthetic construct
  REGION 1..4: note = amino acid sequence added to the carboxy terminus of designed sequences
RRLC

SEQ ID NO: 35 - protein, 23 aa, synthetic construct
  REGION 1..23: note = designed TMD amino acid sequence
LLFILVAILGGLFGAIVAFLLAL

SEQ ID NO: 36 - protein, 23 aa, synthetic construct
  REGION 1..23: note = designed TMD amino acid sequence
LTVALILGIFLGTFIAFWVVYLL

SEQ ID NO: 37 - DNA, 1512 bp, synthetic construct
  misc_feature 1..1512: note = Coding for ProCAR-2 DNA sequence, based on proMP C2.1
  source 1..1512: mol_type = other DNA
atgtctagacaggtacaactgcagcagtcaggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcgaacaaaaactcatctcagaagaggatctgaatggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcggcccggccagcggcggggggcgcagtgcacacgagggggctcgatcccttttggctgactgtggcccttattcttgggatattcttaggaacattcatagcattttgggtcgtttacttgttatgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 38 - protein, 503 aa, synthetic construct
  REGION 1..503: note = Coding for ProCAR-2 amino acid sequence based on proMP C2.1
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLTVALILGIFLGTFIAFWVVYLLWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1512 bp, synthetic construct
  misc_feature 1..1512: note = Coding for ProCAR-3 DNA sequence based on proMP 1.2
  source 1..1512: mol_type = other DNA
atgtctagacaggtacaactgcagcagtcaggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcgaacaaaaactcatctcagaagaggatctgaatggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcggcccggccagcggcggggggcgcagtgcacacgagggggctcgatcccttttggctgttgttcatcctggtggccattttgggtggacttttcggagctatagtcgcatttctcttggccctatgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 40 - protein, 503 aa, synthetic construct
  REGION 1..503: note = ProCAR-3 amino acid sequence based on proMP 1.2
MSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLLFILVAILGGLFGAIVAFLLALWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 69 bp, synthetic construct
  misc_feature 1..69: note = nucleic acid coding for SEQ ID No. 35
  source 1..69: mol_type = other DNA
ttgttatttattctcgtcgcaatacttggaggcttatttggggcgattgttgcattccttttggcgtta

SEQ ID NO: 42 - DNA, 69 bp, synthetic construct
  misc_feature 1..69: note = nucleic acid coding for SEQ ID No. 36
  source 1..69: mol_type = other DNA
ctgaccgtggcgctgattctgggcatcttcctgggcacctttattgcgttttgggtggtgtatctgctg

SEQ ID NO: 43-44 - skipped (2 entries)

SEQ ID NO: 45 - protein, 24 aa, synthetic construct
  REGION 1..24: note = programmed membrane proteins proMPs
  SITE 1: note = misc_feature - Xaa can be P/L/I/V/F/M/S/T/W/Y
  REGION 2..3: note = misc_feature - Xaa can be L/I/V/F/M/S/T/W/Y
  SITE 4: note = misc_feature - Xaa can be F/A/I/L/M/S/V
  SITE 6: note = misc_feature - Xaa can be L/I/V/F/M/S/T/W/Y
  SITE 7: note = misc_feature - Xaa can be V/A/I/L/M/T
  SITE 10: note = misc_feature - Xaa can be L/I/V/F/M/S/T/W/Y
  SITE 12: note = misc_feature - Xaa can be G/A/M/S
  SITE 13: note = misc_feature - Xaa can be L/I/V/F/M/S/T/W/Y
  SITE 14: note = misc_feature - Xaa can be L/I/V/F/M/S/T/W/Y
  SITE 16: note = misc_feature - Xaa can be A/F/I/M/S
  SITE 17: note = misc_feature - Xaa can be I/L/V/F/M/S/T/W/Y
  SITE 18: note = misc_feature - Xaa can be V/L/I/F/M/S/T/W/Y
  SITE 19: note = misc_feature - Xaa can be A/S
  SITE 20: note = misc_feature - Xaa can be F/I/L/M
  REGION 21..22: note = misc_feature - Xaa can be L/I/V/F/M/S/T/W/Y
  SITE 23: note = misc_feature - Xaa can be A/M/S/T/V
  SITE 24: note = misc_feature - Xaa can be L/I/V/F/M/S/T/W/Y
XXXXIXXAIXGXXXGXXXXXXXXX

SEQ ID NO: 46 - protein, 15 aa, synthetic construct
  REGION 1..15: note = programmed membrane proteins proMPs
  REGION 2..3: note = misc_feature - Xaa can be any naturally occurring amino acid
  SITE 6: note = misc_feature - Xaa can be any naturally occurring amino acid
  SITE 8: note = misc_feature - Xaa can be G/A/M/S
  REGION 9..10: note = misc_feature - Xaa can be any naturally occurring amino acid
  REGION 12..14: note = misc_feature - Xaa can be any naturally occurring amino acid
  SITE 15: note = misc_feature - Xaa can be A/S
IXXAIXGXXXGXXXX

SEQ ID NO: 47-54 - skipped (8 entries)

SEQ ID NO: 55 - protein, 24 aa, synthetic construct
  REGION 1..24: note = programmed membrane proteins proMPs
  SITE 1: note = misc_feature - Xaa can be P/L/I/V/F/M/S/T/W/Y
  SITE 2: note = misc_feature - Xaa can be I/L/V/F/M/S/T/W/Y
  SITE 3: note = misc_feature - Xaa can be L/I/V/F/M/S/T/W/Y
  SITE 4: note = misc_feature - Xaa can be I/L
  SITE 6: note = misc_feature - Xaa can be L/I/V/F/M/S/T/W/Y
  SITE 7: note = misc_feature - Xaa can be A/F/I/L/M/S/V
  SITE 9: note = misc_feature - Xaa can be L/I/V/F/M/S/T/W/Y
  SITE 11: note = misc_feature - Xaa can be G/A
  SITE 12: note = misc_feature - Xaa can be A/M/S
  SITE 13: note = misc_feature - Xaa can be V/L/I/F/M/S/T/W/Y
  SITE 14: note = misc_feature - Xaa can be I/L/V/F/M/S/T/W/Y
  SITE 16: note = misc_feature - Xaa can be A/F/I/L/M/S/T/V
  SITE 17: note = misc_feature - Xaa can be I/L/V/F/M/S/T/W/Y
  SITE 19: note = misc_feature - Xaa can be S/A/F/I/L/M/T/V
  SITE 20: note = misc_feature - Xaa can be F/L/I/V/M/S/T/W/Y
  REGION 21..22: note = misc_feature - Xaa can be F/L/I/V/M/S/T/W/Y
  SITE 23: note = misc_feature - Xaa can be V/A/F/I/L/M/S/T
  SITE 24: note = misc_feature - Xaa can be Y/L/I/V/M/S/T/W/F
XXXXTXXMXTXXXXGXXSXXXXXX

SEQ ID NO: 56 - protein, 15 aa, synthetic construct
  REGION 1..15: note = programmed membrane proteins proMPs
  SITE 1: note = misc_feature - Xaa can be I/L
  REGION 3..4: note = misc_feature - Xaa can be any naturally occurring amino acid
  SITE 6: note = misc_feature - Xaa can be any naturally occurring amino acid
  SITE 8: note = misc_feature - Xaa can be G/A
  SITE 9: note = misc_feature - Xaa can be A/M/S
  REGION 10..11: note = misc_feature - Xaa can be any naturally occurring amino acid
  REGION 13..14: note = misc_feature - Xaa can be any naturally occurring amino acid
XTXXMXTXXXXGXXS

SEQ ID NO: 57-60 - skipped (4 entries)

SEQ ID NO: 61 - protein, 24 aa, synthetic construct
  REGION 1..24: note = programmed membrane proteins proMPs
  SITE 1: note = misc_feature - Xaa can be P/L/I/V/F/M/S/T/W/Y
  SITE 2: note = misc_feature - Xaa can be T/A/I/L/M/S/V
  SITE 3: note = misc_feature - Xaa can be V/L/I/F/M/S/T/W/Y
  SITE 5: note = misc_feature - Xaa can be S/A/F/L/V
  SITE 6: note = misc_feature - Xaa can be A/F/L/M/S/T
  SITE 7: note = misc_feature - Xaa can be I/L/V/F/M/S/T/W/Y
  SITE 8: note = misc_feature - Xaa can be L/I/V/F/M/S/T/W/Y
  SITE 10: note = misc_feature - Xaa can be V/A/F/I/L/M/S/T
  SITE 11: note = misc_feature - Xaa can be S/L/I/V/F/M/S/T/W/Y
  SITE 12: note = misc_feature - Xaa can be F/L/I/V/M/S/T/W/Y
  SITE 14: note = misc_feature - Xaa can be T/A/F/G/I/L/M/S/V
  SITE 15: note = misc_feature - Xaa can be F/L/I/V/M/S/T/W/Y
  SITE 16: note = misc_feature - Xaa can be I/F/L/V
  SITE 17: note = misc_feature - Xaa can be S/A/G/L/M/T/V
  SITE 18: note = misc_feature - Xaa can be L/I/V/F/M/S/T/W/Y
  SITE 19: note = misc_feature - Xaa can be V/A/F/I/L/M/S
  SITE 21: note = misc_feature - Xaa can be L/I/V/F/M/S/T/W/Y
  SITE 22: note = misc_feature - Xaa can be W/L/I/V/F/M/S/T/Y
  SITE 23: note = misc_feature - Xaa can be L/I/V/F/M/S/T/W/Y
  SITE 24: note = misc_feature - Xaa can be A/G/S
XXXTXXXXGXXXGXXXXXXAXXXX

SEQ ID NO: 62 - protein, 21 aa, synthetic construct
  REGION 1..21: note = programmed membrane proteins proMPs
  SITE 2: note = misc_feature - Xaa can be S/A/F/L/V
  REGION 3..5: note = misc_feature - Xaa can be any naturally occurring amino acid
  REGION 7..9: note = misc_feature - Xaa can be any naturally occurring amino acid
  REGION 11..12: note = misc_feature - Xaa can be any naturally occurring amino acid
  SITE 13: note = misc_feature - Xaa can be I/F/L/V
  REGION 14..16: note = misc_feature - Xaa can be any naturally occurring amino acid
  REGION 18..20: note = misc_feature - Xaa can be any naturally occurring amino acid
  SITE 21: note = misc_feature - Xaa can be A/G/S
TXXXXGXXXGXXXXXXAXXXX

SEQ ID NO: 63-68 - skipped (6 entries)

SEQ ID NO: 69 - protein, 24 aa, synthetic construct
  REGION 1..24: note = programmed membrane proteins proMPs
  SITE 1: note = misc_feature - Xaa can be T/A/F/G/I/L/M/S/V
  SITE 2: note = misc_feature - Xaa can be A/G/S
  SITE 3: note = misc_feature - Xaa can be L/I
  SITE 4: note = misc_feature - Xaa can be L/A/F/I/M/S/T/V/W
  SITE 5: note = misc_feature - Xaa can be V/A/F/I/L/M/S/T/W/Y
  SITE 7: note = misc_feature - Xaa can be F/A/I/K/L/M/R/V
  SITE 8: note = misc_feature - Xaa can be V/A/F/I/L/M/S/T/W/Y
  SITE 10: note = misc_feature - Xaa can be Y/A/F/I/K/L/M/S/T/V/W
  SITE 11: note = misc_feature - Xaa can be Y/A/F/G/I/L/M/S/T/V/W
  SITE 12: note = misc_feature - Xaa can be T/A//F/I/L/M/S/V/W/Y
  SITE 14: note = misc_feature - Xaa can be L/I/V
  SITE 15: note = misc_feature - Xaa can be I/A/F/L/M/T/V
  SITE 16: note = misc_feature - Xaa can be A/G
  SITE 17: note = misc_feature - Xaa can be L/F
  SITE 18: note = misc_feature - Xaa can be I/F/L/M/V/W
  SITE 19: note = misc_feature - Xaa can be F/A/I/L/M/S/T/V/W/Y
  SITE 21: note = misc_feature - Xaa can be I/A/L/M/S/V
  SITE 22: note = misc_feature - Xaa can be L/A/I/W
  SITE 23: note = misc_feature - Xaa can be A/S
  SITE 24: note = misc_feature - Xaa can be T/A/F/G/I/L/M//V/W/Y
XXXXXAXXAXXXAXXXXXXAXXXX

SEQ ID NO: 70 - protein, 22 aa, synthetic construct
  REGION 1..22: note = programmed membrane proteins proMPs
  SITE 1: note = misc_feature - Xaa can be A/G/S
  SITE 2: note = misc_feature - Xaa can be L/I
  REGION 3..4: note = misc_feature - Xaa can be any naturally occurring amino acid
  REGION 6..7: note = misc_feature - Xaa can be any naturally occurring amino acid
  REGION 9..11: note = misc_feature - Xaa can be any naturally occurring amino acid
  SITE 13: note = misc_feature - Xaa can be L/I/V
  SITE 14: note = misc_feature - Xaa can be any naturally occurring amino acid
  SITE 15: note = misc_feature - Xaa can be A/G
  SITE 16: note = misc_feature - Xaa can be L/F
  REGION 17..18: note = misc_feature - Xaa can be any naturally occurring amino acid
  REGION 20..21: note = misc_feature - Xaa can be any naturally occurring amino acid
  SITE 22: note = misc_feature - Xaa can be A/S
XXXXAXXAXXXAXXXXXXAXXX

SEQ ID NO: 71 - protein, 24 aa, synthetic construct
  REGION 1..24: note = proMP C4.1amino acid sequence
PLLVALLALLAVIAALLAAIFALL

SEQ ID NO: 72-73 - skipped (2 entries)

SEQ ID NO: 74 - DNA, 72 bp, synthetic construct
  misc_feature 1..72: note = proMP C4.1 nucleic acid sequence
  source 1..72: mol_type = other DNA
ccccttctcgtagcattgctggcccttctggccgtcatagctgccctgctggccgcaatttttgccctgctc

SEQ ID NO: 75 - DNA, 66 bp, synthetic construct
  misc_feature 1..66: note = new sequence for proMPC4.1 (used in proCAR-4), which is seq ID 15 with the first and last amino acids deleted
  source 1..66: mol_type = other DNA
cttctcgtagcattgctggcccttctggccgtcatagctgccctgctggccgcaatttttgccctg

SEQ ID NO: 76 - DNA, 1563 bp, synthetic construct
  misc_feature 1..1563: note = Coding for ProCAR-4 DNA sequence based on proMP C4.1
  source 1..1563: mol_type = other DNA
atggattttcaggtgcagattttcagcttcctgctaatcagtgcctcagtcataatgtctagacaggtacaactgcagcagtcaggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcgaacaaaaactcatctcagaagaggatctgaatggggtcaccgtctcttcagcgctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcggcccggccagcggcggggggcgcagtgcacacgagggggctcgatcccttttggcttctcgtagcattgctggcccttctggccgtcatagctgccctgctggccgcaatttttgccctgtgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 77 - protein, 520 aa, synthetic construct
  REGION 1..520: note = ProCAR-4 amino acid sequence based on proMP C4.1
MDFQVQIFSFLLISASVIMSRQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIEQKLISEEDLNGVTVSSALSNSIMYFSHFVPVFLPAKPTTTPAPRPPTPAPTIASQPLSLRPEAARPAAGGAVHTRGLDPFWLLVALLALLAVIAALLAAIFALWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0201257 A1
(43) Pub. Date: Jun. 29, 2023

(54) DE-NOVO DESIGNED TRANSMEMBRANE POLYPEPTIDES AND THEIR USES IN CELLULAR IMMUNOTHERAPY

(71) Applicants: Yeda Research and Development Co. Ltd., Rehovot (IL); The Walter and Eliza Hall Institute of Medical Research, Victoria (AU)

(72) Inventors: Sarel FLEISHMAN, Rehovot (IL); Assaf ELAZAR, Rehovot (IL); Jonathan Y. WEINSTEIN, Rehovot (IL); Melissa J. CALL, Victoria (AU); Matthew E. CALL, Victoria (AU); Nicholas J. CHANDLER, Victoria (AU)

(73) Assignees: Yeda Research and Development Co. Ltd., Rehovot (IL); The Walter and Eliza Hall Institute of Medical Research, Victoria (AU)

(21) Appl. No.: 17/986,080

(22) Filed: Nov. 14, 2022

Related U.S. Application Data
(63) Continuation of application No. PCT/IL2021/050556, filed on May 13, 2021.

(30) Foreign Application Priority Data
May 14, 2020 (IL) 274696

(51) Int. Cl. A61K 35/17; C07K 14/725; C07K 14/705; C07K 16/32; A61P 35/00

(52) U.S. Cl. CPC A61K 35/17; A61P 35/00; C07K 14/7051; C07K 14/70517; C07K 14/70521; C07K 16/32; C07K 2317/622; C07K 2319/03; C07K 2319/41

(57) ABSTRACT

Provided herein is a family of programmable de novo designed transmembranal protein domain polypeptides, and uses thereof in the production of engineered chimeric antigen receptor T (CAR T) cells, which are used, for example, to fight cancer.

### RELATED APPLICATIONS

This application is a Continuation of PCT Patent Application No. PCT/IL2021/050556 filed on May 13, 2021, which claims the benefit of priority of Israel Patent Application No. 274696 filed on May 14, 2020. The contents of the above applications are all incorporated by reference as if fully set forth herein in their entirety.

### SEQUENCE LISTING STATEMENT

The XML file, entitled 94521SequenceListing.xml, created on Nov. 14, 2022, comprising 124,618 bytes, submitted concurrently with the filing of this application is incorporated herein by reference.

### FIELD AND BACKGROUND OF THE INVENTION

The present invention, in some embodiments thereof, relates to protein design, and more particularly, but not exclusively, to de-novo designed transmembrane protein chains.

The α-helical transmembrane domains (TMDs) of single-pass receptors couple ligand binding to signal initiation across the cell membrane and control receptor function by altering the receptors’ oligomeric state or conformation. Design of stable and specific TMDs thus offers an attractive way to probe structure-activity relationships in natural receptors and to tailor engineered receptor function, but progress towards de-novo receptor TMD design has been elusive. Interactions among cell-surface receptors play central roles in determining complex receptor structures and controlling signal propagation. In immune receptors, death receptors and growth factor receptors, for example, the transmembrane domains (TMDs) govern key interactions involved in assembly, activation and higher-order clustering. Control over the specificity, stability, geometry and oligomeric state of these interactions is highly desirable both for mechanistic studies of natural receptors and in the engineering of synthetic receptors. Such control, however, is difficult to achieve using natural TMDs that have likely been evolutionarily selected for a degree of flexibility in these very attributes. The importance of high precision in receptor engineering has come into particularly sharp focus with the recent FDA approval of the first cellular immunotherapies for cancer using chimeric antigen receptors (CARs), single-chain hybrid sensors that endow polyclonal patient T cells with cytotoxic antitumor activity.

The modular domain structure of CARs offers significant scope to incorporate highly customized sequences into the antigen-binding, hinge, TMD and/or signaling domains that could modulate receptor structure and CAR-T cell function in predictable ways. The TMDs, however, have received little attention in systematic studies of CAR design. For convenience, most investigations have used the TMD sequence of the same protein from which the adjacent hinge or signaling domains were derived; that is, mostly from endogenous T cell proteins such as CD4, CD8α, CD28 or the T cell receptor (TCR)-associated CD3ζ chain. These sequences can engage in molecular interactions that drive self-association and/or assembly with their endogenous T cell counterparts and impact CAR surface expression and functional properties in ways that reduce control over signaling outcomes. The CD3ζ TMD, for example, drives both self-association (to form homodimers) and incorporation into endogenous TCR-CD3 complexes in the T-cell membrane. The sequence determinants of these homotypic and heterotypic interactions are overlapping and therefore difficult to disentangle, and yet both appear to exert significant control over CAR signaling. The TMD sequences used in current FDA-approved CAR-T cell therapies derive from CD8α and CD28; although experimentally less well characterized, these sequences are likely to carry similar risks of confounding interactions with endogenous T cell signaling proteins.

Fleishman, S. J. et al. [“A putative molecular-activation switch in the transmembrane domain of ErbB2”, Proc. Natl. Acad. Sci. U. S.A., 2002, 99, 15937-15940] proposed a molecular mechanism for rotation-coupled activation of the receptor tyrosine kinase (RTK) ErbB2 (also designated neu or HER2). Using a computational exploration of conformation space of the transmembrane (TM) segments of an ErbB2 homodimer, the authors found two stable conformations of the TM domain, and suggest that these conformations correspond to the active and inactive states of ErbB2, and that the receptor molecules may switch from one conformation to the other without crossing exceedingly unfavorable states. The proposed model provides an explanation for the biochemical and oncogenic properties of ErbB2, such as the effects of ErbB2 overexpression on kinase activity and cell transformation. Furthermore, the opposing effects of the neu* activating oncogenic point mutation and the Val-655-»lle single-nucleotide polymorphism shown to be linked to reduced risk of breast cancer are explained in terms of shifts in the equilibrium between the active and inactive states of ErbB2 in vivo.

Barth, P. and Senes, A. [“Toward high-resolution computational design of the structure and function of helical membrane proteins”, Nat. Struct. Mol. Biol., 2016, 23, 475-480] reported that the computational design of α-helical membrane proteins is still in its infancy but has already made great progress. De novo design allows stable, specific and active minimal oligomeric systems to be obtained. Computational reengineering can improve the stability and function of naturally occurring membrane proteins. The authors reported that the major hurdle for the field is the experimental characterization of the designs, and that the emergence of new structural methods for membrane proteins will accelerate progress.

Korendovych, I.V. and De-Grado, W.F. [“De novo protein design, a retrospective”, Q. Rev. Biophys., 2020, 53, e3] review the evolution of the field of de novo protein design from its earliest days, placing particular emphasis on how this endeavor has illuminated our understanding of the principles underlying the folding and function of natural proteins, and is informing the design of macromolecules with unprecedented structures and properties.

Weinstein, J. Y., Elazar, A. and Fleishman, S. J. [“A lipophilicity-based energy function for membrane-protein modelling and design”, PLoS Comput. Biol., 2019, 15, e1007318] have relied on a previously described high-throughput experimental screen, called dsTβL, that inferred apparent insertion energies for each amino acid at dozens of positions across the bacterial plasma membrane [Elazar, A. et al., “Mutational scanning reveals the determinants of protein insertion and association energetics in the plasma membrane”, eLife 2016; 5. pii: e12125]. Weinstein et al have expressed these profiles as lipophilicity energy terms in Rosetta and have demonstrated that the new energy function outperforms previous ones in modelling and design benchmarks. Rosetta ab initio simulations starting from an extended chain recapitulate two-thirds of the experimentally determined structures of membrane-spanning homo-oligomers with less than 2.5 Å root-mean-square deviation within the top-predicted five models. It is further shown, in two sequence-design benchmarks, that the energy function improves discrimination of stabilizing point mutations and recapitulates natural membrane-protein sequences of known structure, thereby recommending this new energy function for membrane-protein modelling and design.

### SUMMARY OF THE INVENTION

The present invention provides and validates a strategy for de novo design and structural characterization of programmed membrane proteins (proMPs): single-pass α-helical TMDs that form ultra-stable oligomeric complexes through atomically accurate computationally defined interfaces. To demonstrate the usefulness of proMPs, the present inventors have programed specific homotypic interactions in HER2-specific chimeric antigen receptors (to generate proCARs containing an anti-HER2 scFv, human CD8α stalk sequence, a proMP TMD, human CD28 co-stimulatory sequence and human ζ-chain signalling tail) without altering key functional domains in the extracellular and intracellular spaces. CAR-T cells expressing dimeric or trimeric proCARs exhibit enhanced target-cell cytotoxicity in vitro compared to a reference CAR (identical but for the human CD28 TMD sequence) similar to those currently used in the clinic. Additionally, all proCARs tested show strongly attenuated inflammatory cytokine release in vitro. In a mouse model of HER2+ cancer, anti-tumor activity increased with oligomeric state, with the trimeric proCAR most closely approaching the efficacy of the reference CD28 TMD-containing CAR. The proCAR platform therefore provides predictable control of CAR-T cell potency on a background of attenuated cytokine release, suggesting that the orthogonal structural control achieved through proMPs could provide improvements in both safety and efficacy of cellular immunotherapies for cancer and other diseases by widening the therapeutic window. Thus, the proMPs provided herein afford an exceptionally modular route to tuning engineered receptor function and can be easily incorporated into existing CAR designs.

Thus, according to an aspect of some embodiments of the present invention, there is provided a designed transmembrane domain polypeptide including or consisting an amino-acid sequence selected from the group consisting of any one of the sequences corresponding to motifs 1-15, SEQ ID No. 35 and SEQ ID No. 36, and SEQ ID No. 71, wherein:
the sequences corresponding to motif 1 are presented in Table 1;
the sequences corresponding to motif 2 are presented in Table 2;
the sequences corresponding to motif 3 are presented in Table 3;
the sequences corresponding to motif 4 are presented in Table 4;
the sequences corresponding to motif 5 are presented in Table 5;
the sequences corresponding to motif 6 are presented in Table 6;
the sequences corresponding to motif 7 are presented in Table 7;
the sequences corresponding to motif 8 are presented in Table 8;
the sequences corresponding to motif 9 are presented in Table 9;
the sequences corresponding to motif 10 are presented in Table 10;
the sequences corresponding to motif 11 are presented in Table 11;
the sequences corresponding to motif 12 are presented in Table 12;
the sequences corresponding to motif 13 are presented in Table 13;
the sequences corresponding to motif 14 are presented in Table 14; and
the sequences corresponding to motif 15 are presented in Table 15.

In some embodiments, the amino-acid sequence of the designed transmembrane domain polypeptide is:
the amino-acid sequence corresponding to motif 1 begins at position 7 and ends at position 22, and having a sequence G/A-XXX-A/S-XX-G/A-XXX-G-XXX-A/S;
the amino-acid sequence corresponding to motif 2 begins at position 5 and ends at position 19, and having a sequence I-XX-AI-X-G-G/A/M/S-XX-G-XXX-A/S;
the amino-acid sequence corresponding to motif 3 begins at position 6 and ends at position 16, and having a sequence I-X-A/M/S-A/M/S-XX-G/A-A/S-XX-A/M/S/T;
the amino-acid sequence corresponding to motif 4 begins at position 7 and ends at position 22, and having a sequence G/A-XXX-A-XX-A/M/S-XXX-G-XXX-S/A/F/M/T-XXX-L/I;
the amino-acid sequence corresponding to motif 5 begins at position 6 and ends at position 19, and having a sequence L/I/V-XXXXXXX-A/F/M/S/T-A/F/M/S/T-XXX-L/I/V;
the amino-acid sequence corresponding to motif 6 begins at position 7 and ends at position 23, and having a sequence L/A/M/S-XXX-G/A-XXX-G-A/F/S-XX-A-XXX-A/T/V-XXX-L/F/II-XX-AI-X-G-G/A/M/S-XX-G-XXX-A/S;
the amino-acid sequence corresponding to motif 7 begins at position 4 and ends at position 18, and having a sequence I/L-T-XX-M-X-T-G/A-A/M/S-XX-G-XX-S;
the amino-acid sequence corresponding to motif 8 begins at position 2 and ends at position 20, and having a sequence A-XX-I/L/T/V-X-L/I-X-I/F/L-XXX-FI-A/S-XXX-G/A-L/I;
the amino-acid sequence corresponding to motif 9 begins at position 3 and ends at position 10, and having a sequence A/F/M/S-XX-L/I-XXX-A/S/T;
the amino-acid sequence corresponding to motif 10 begins at position 4 and ends at position 24, and having a sequence T-S/A/F/L/V-XXX-G-XXX-G-XX-I/F/L/V-XXX-A-XXX-A/G/S;
the amino-acid sequence corresponding to motif 11 begins at position 9 and ends at position 20, and having a sequence I/F/L-VL-XXXX-G/A-XXX-A;
the amino-acid sequence corresponding to motif 12 begins at position 10 and ends at position 22, and having a sequence A/F/M/S-XXX-G/A/F/L/M/S-A/F/I/M/S/V-X-I/F/L/M-XXXX-A/T/V;
the amino-acid sequence corresponding to motif 13 begins at position 5 and ends at position 17, and having a sequence A/F/S-L/I-I-X-G-I/A/M/T/V-XX-G-XXX-A/S/V;
the amino-acid sequence corresponding to motif 14 begins at position 2 and ends at position 23, and having a sequence A/G/S-L/I-XX-A-XX-A-XXX-A-L/I/V-X-A/G-L/F-XX-A-XX-A/S;
the amino-acid sequence corresponding to motif 15 begins at position 7 and ends at position 18, and having a sequence L-A/G/S-XX-A/G/S-XXX-A/G/S-XX-A/S;
and “X” is optionally selected from the group of L, I, V, F, M, S, T, W, and Y.

According to an aspect of some embodiments of the present invention, there is provided a polypeptide sequence, selected from the group consisting of any combination of any amino acid per entry as presented in Tables 1-15, SEQ ID No. 35 and SEQ ID No. 36 and SEQ ID No. 72.

According to another aspect of some embodiments of the present invention, there is provided a CAR-T cell that includes at least one of the polypeptides having at least one of the sequences presented in Tables 1-15, SEQ ID No. 35 and SEQ ID No. 36 and SEQ IS No. 72.

According to another aspect of some embodiments of the present invention, there is provided a CAR-T cell engineered to express a CAR polypeptide including or consisting of SEQ ID No. 38, SEQ ID No. 40, or SEQ ID No. 77.

In some embodiments, the CAR-T cell provided herein is for use in the treatment of cancer and/or adoptive cell therapy.

According to another aspect of some embodiments of the present invention, there is provided a pharmaceutical composition including at least one of the CAR-T cell presented herein, and a pharmaceutically acceptable carrier.

In some embodiments, the pharmaceutical composition provided herein is for use in the treatment of cancer and/or adoptive cell therapy.

According to another aspect of some embodiments of the present invention, there is provided a method of treating a subject in need of, which is effected by administering to the subject a therapeutically effective amount of the pharmaceutical composition provided herein.

In some embodiments, the method provided herein is for use in the treatment of cancer and/or adoptive cell therapy.

According to another aspect of some embodiments of the present invention, there is provided a use of at least one of polypeptide having the sequence presented in Tables 1-15, SEQ ID No. 35 and SEQ ID No. 36 and SEQ ID No. 72, in the production of engineered CAR-T cells.

In some embodiments of the present invention, the CAR-T cell presented herein, or the pharmaceutical composition presented herein, or the method presented herein, is suitable for use in the treatment of cancer and/or adoptive cell therapy.

According to another aspect of some embodiments of the present invention, there is provided a designed transmembrane domain polypeptide, including any one of the herein-presented motifs 1-15.

In some embodiments of the present invention, the motif is selected from the group consisting of motifs 2, and 13-15.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C present results of design round 1 that generated a trimer, wherein FIG. 1A presents a gel running three different proMPs designed to form homodimers that were expressed and purified as free peptides, and proMP 1.2 (SEQ ID NO. 2) appeared to migrate as a stable trimer by reducing SDS-PAGE, FIG. 1B shows a 2.55 Å structure of proMP 1.2 (SEQ ID NO. 2) determined by X-ray diffraction from crystals grown in monoolein lipid cubic phase that confirmed a trimeric structure, and FIG. 1C shows the ab initio generated model in C3 symmetry that aligned well with the experimental structure;

FIGS. 2A-K present results of design round 2 showing that the designs accurately form target oligomeric states, wherein ProMP C2.1 (SEQ ID No. 13; FIGS. 2A-B) and proMP C3.1 (SEQ ID No. 14; FIGS. 2C-D) yielded structures from crystals grown in monoolein lipid cubic phase (proMP C2.1; SEQ ID No. 13; 2.7 Å resolution) or in C5E8 detergent (proMP C3.1; SEQ ID No. 14; 3.5 Å resolution), proMP C2.1 (SEQ ID No. 13; FIGS. 2E-G) structure aligned to the design model with 0.3 Å RMSD in the helix packing interface, proMP C3.1 (SEQ ID No. 14; FIGS. 2F-K) aligned to the design model with 0.4 Å RMSD for two of the helices (FIG. 21) but the third helix was antiparallel (FIG. 2J) and used a packing arrangement of alanine residues that was nearly identical to packing model but in reverse (FIG. 2K);

FIGS. 3A-E demonstrate that HER2 proCARs incorporating dimeric and trimeric proMP sequences provide enhanced cytotoxicity and attenuated cytokine release in vitro, wherein FIG. 3A showns a 4-hour chromium-51 release assay was performed to measure cytotoxic killing of mouse MC57-HER2 tumor cells by mouse CD8+ CAR-T cells, P values were determined using unpaired t-tests comparing Reference and proCAR-T cell data at 20:1 effector to target cell (E:T) ratio from 3 independent experiments, FIGS. 3B-E show that CAR-T cells were co-cultured with MC57-HER2 target cells for 24 hours and the indicated cytokines were measured in culture supernatants using a cytokine bead array (CBA) assay. Statistical significance (*p<0.05; **p<0.01; ***p<0.005) was determined using paired t-tests;

FIGS. 4A-F demonstrate that HER2 proCAR incorporating tetrameric design proMP C4.1 provide robust cytotoxicity and attenuated cytokine release in vitro, wherein FIG. 4A shows proMP C4.1 tetrameric design model, FIG. 4B shows HER2 ProCAR-4 T cells were compared to proCAR-3, Reference and No CAR T cells in a live-cell imaging (Incucyte) assay over 24 hours at low (1:2) effector to target cell (E:T) ratio, and that differences in killing curves were not significantly different in a 2-way ANOVA performed on three replicate data sets, and FIGS. 4C-F show cytokine release measured as in FIGS. 3A-E, whereas data are from one experiment with two replicate measurements; and

FIGS. 5A-B show that proCAR-T cells provide in vivo tumor control that scales with oligomeric state, wherein FIG. 5A shows that female NSG mice at 6-8 weeks of age (5-6 per group) were engrafted with mouse MC38-HER2 tumor cells (5×10⁵ s.c.) and 10⁷ CAR-T cells were delivered i.v. one day later (tumor growth was monitored daily using calipers)(symbols represent the mean and standard error for each group, repeated measures 2-way ANOVA test, *p<0.05, **p<0.01), and FIG. 5B presents a linear regression through the means of measurements shown in FIG. 5A show statistically significant association of tumour control with oligomeric state.

### DESCRIPTION OF SOME SPECIFIC EMBODIMENTS OF THE INVENTION

The present invention, in some embodiments thereof, relates to de-novo designed proteins, and more particularly, but not exclusively, to a list of de-novo designed transmembrane protein chains.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Design Rationale

The present inventors have set out to define the relationships between TMD structure and signaling in CAR-T cells. To achieve this goal, the inventors have contemplated the design of completely new TMDs with programmable self-association features and minimal risk of crosstalk with other native T-cell components. Despite significant recent progress in the field, the limitations of membrane-protein (MP) atomistic calculations have restricted de novo MP design studies to highly predictable and rigid coiled-coil motifs that, while stabilizing them, limited their usefulness as receptor TMDs. By contrast, the inventors have described an accurate ab initio Rosetta atomistic modelling strategy [Weinstein, J. Y., Elazar, A. and Fleishman, S. J., PLoS Comput. Biol., 2019, 15, e1007318] for single-spanning homo-oligomers using a new energy function with experimentally determined membrane-solvation terms for each amino acid.

While reducing the present invention to practice, the inventors have implemented a new strategy to de novo design proMPs (programmable Membrane Proteins) — stable TM homo-oligomers of defined geometry and order that can be used to program cell-surface receptor structure. Structure-validated proMPs were used to generate proCAR constructs, and the results have shown that these endowed T cells with robust cytotoxicity and lower inflammatory cytokine release compared to otherwise identical CARs containing natural TMDs. These results shed new light on the importance of precision in engineered receptor structure and intermolecular associations for optimal CAR-T activity.

### Initial Design Approach

In the initial design approach, each computational trajectory started from two fully symmetric and extended chains of 24 amino acids encoding either poly-Val or poly-Ala. In a first, coarse-grained modelling step, backbone torsion angles were sampled from a database comprising 3 and 9 amino acid fragments from α-helical MPs, and the two chains were symmetrically docked against one another with an energy term that disfavored large crossing angles. In a second, all-atom step, the sequence and the structure were refined through iterations of symmetric sequence optimization, backbone minimization, and rigid-body docking using the ref2015_memb atomistic energy function that is dominated by van der Waals packing, hydrogen bonding and amino acid lipophilicity. It had been noticed that the resulting sequences were overwhelmingly biased towards the large and flexible hydrophobic amino acid Leu, as expected from the dominant role of lipophilicity in the ref2015_memb potential. Forward-folding ab initio structure-prediction calculations, however, indicated that the designs were prone to form multiple alternative low-energy dimers instead of the designed ones.

To mitigate the risk of misfolding due to the high Leu content, a sequence diversification step was introduced, comprising 120 iterations of single-point mutations and energy relaxation while biasing the sequence composition to match that of natural TMDs. The resulting sequences were subjected to ab initio structure prediction calculations and this time, they converged to the design models and exhibited a large energy gap from undesired structures. It was noted that natural TMDs are not optimized for thermodynamic stability; interestingly, our results suggest that evolution may have selected sequence compositions to counter TMD misfolding.

### Designed Transmembranal Polypeptides

As a proof of concept, 12 de novo designed proteins were cloned, expressed and tested in the E. coli TOXCAT-β-lactamase (TβL) selection system. In this dual-reporter system, survival on ampicillin and chloramphenicol reports on a design’s membrane-insertion and self-association propensity, respectively. Remarkably, most proMPs designed according to some embodiments of the present invention, supported high survival and two-thirds survived even at the highest chloramphenicol concentration tested, indicating a self-association strength significantly greater than the TMD from ErbB2, which served as a positive control. The patterns of sensitivity to interface mutations of most designs were also consistent with the design models, suggesting that they indeed assembled through the designed interfaces in the bacterial inner membrane.

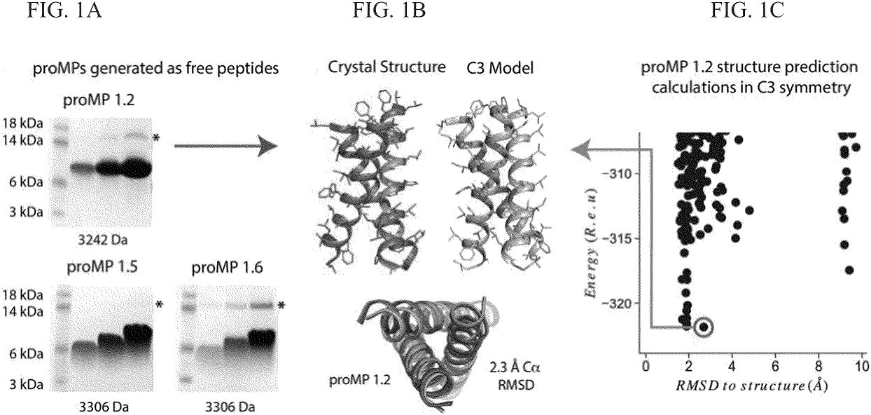

FIGS. 1A-C present results of design round 1 that generated a trimer, wherein FIG. 1A presents a gel running three different proMPs designed to form homodimers that were expressed and purified as free peptides, and proMP 1.2 (SEQ ID NO. 2) appeared to migrate as a stable trimer by reducing SDS-PAGE, FIG. 1B shows a 2.55 Å structure of proMP 1.2 (SEQ ID NO. 2) determined by X-ray diffraction from crystals grown in monoolein lipid cubic phase that confirmed a trimeric structure, and FIG. 1C shows the ab initio generated model in C3 symmetry that aligned well with the experimental structure.

Some proMPs were produced in recombinant methods as free TM peptides, and all exhibited electrophoretic mobility consistent with SDS- and heat-stable self-association; however, the patterns of gel migration were not uniform. Six proMPs had the apparent molecular weight of a dimer (e.g., proMP 1.5 and 1.6; FIG. 1A) and exhibited reduced mobility as the peptide concentration was increased, similar to the SDS-stable behavior of the well-studied glycophorin A TMD. By contrast, the remaining two proMPs exhibited migration that was independent of the sample concentration and had apparent molecular weights more consistent with oligomers larger than the designed dimers (based on proMP 1.2 (SEQ ID No. 2); FIG. 1A). To establish the molecular structure of these designs, several were screened for crystallization in monoolein lipid cubic phase, and the structure of proMP 1.2 (SEQ ID No. 2) was determined to 2.55 Å resolution (FIG. 1B). It is noted herein that polypeptides that were used for gel analysis and crystallization were altered for practical purposes and are therefore not identical to the sequences of the TMD that stem from the method of designing the same. For example, the first proline was deleted and 6 extra amino acids were added, 3 before and 3 after the designed sequence.

While the positions involved in helix packing recapitulated the design model, this proMP indeed formed a trimer instead of the intended dimer. Ab initio structure prediction calculations in trimeric (C3) symmetry recapitulated this packing interface (RMSD 2.3 Å; FIGS. 1B-C), demonstrating that it would have been possible to predict this outcome had alternative oligomeric states would be considered.

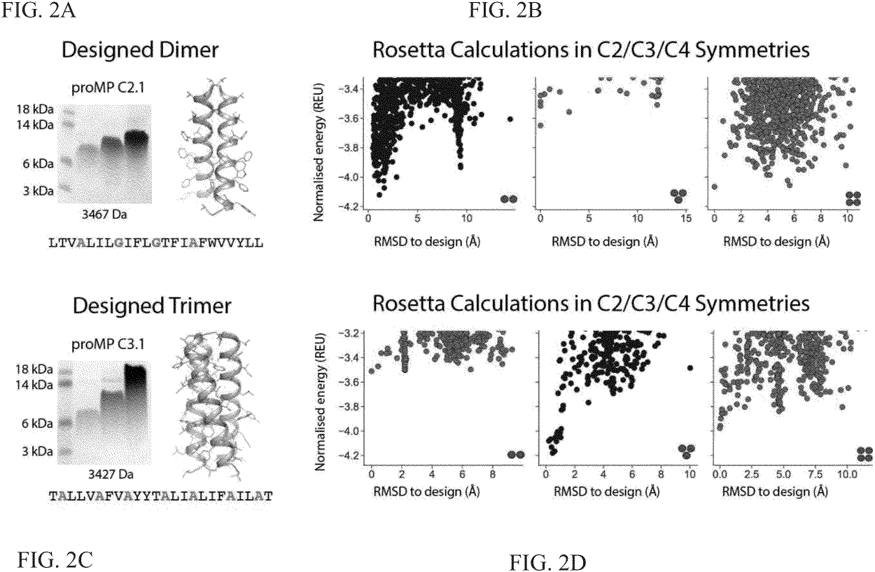

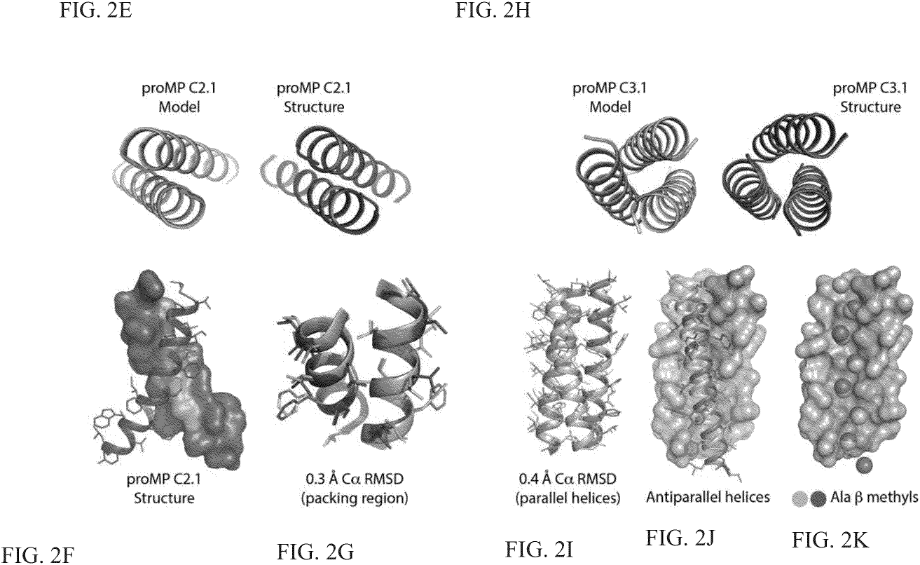

FIGS. 2A-K present results of design round 2 showing that the designs accurately form target oligomeric states, wherein ProMP C2.1 (SEQ ID No. 13; FIGS. 2A-B) and proMP C3.1 (SEQ ID No. 14; FIGS. 2C-D) yielded structures from crystals grown in monoolein lipid cubic phase (proMP C2.1; SEQ ID No. 13; 2.7 Å resolution) or in C5E8 detergent (proMP C3.1; SEQ ID No. 14; 3.5 Å resolution), proMP C2.1 (SEQ ID No. 13; FIGS. 2E-G) structure aligned to the design model with 0.3 Å RMSD in the helix packing interface, proMP C3.1 (SEQ ID No. 14; FIGS. 2F-K) aligned to the design model with 0.4 Å RMSD for two of the helices (FIG. 21) but the third helix was antiparallel (FIG. 2J) and used a packing arrangement of alanine residues that was nearly identical to packing model but in reverse (FIG. 2K).

Based on the foregoing, a third design campaign was initiated to produce proMPs in a range of oligomeric states, incorporating a final step in which ab initio structure prediction calculations were performed in C2 (180° rotation), C3 (120° rotation) and C4 (90° rotation) symmetries for every design (FIGS. 2A-D). Only those proMPs that were predicted to form the target oligomeric state and none of the alternatives were selected for further analysis. This strategy yielded two proMPs for which crystal structures were obtained, confirming the target oligomeric state: a dimer with glycine-based packing (based on proMP C2.1; FIGS. 2E-G), and a trimer with an alanine-rich interface (based on proMP C3.1; FIGS. 2H-K). Interestingly, while two of the helices in the crystal structure of proMP C3.1 aligned well with the design model, the third was in an antiparallel orientation (FIG. 2J). Despite this arrangement, the six key interface alanine β-methyls were in near-identical positions to their counterparts in the fully parallel model (FIG. 2K) leading to the assumption that the model is correct but the crystal lattice was enforcing the antiparallel binding mode of the third helix.

To probe this possibility, the parallel model was aligned with the asymmetric unit seen in the crystal structure and generated crystallographic symmetry, showing clashes for the third helix and indicating that the design model cannot be accommodated in the crystal lattice. The structure thus suggests that this proMP is unintentionally “reversible” in that one of the helices can form the intended packing mode in either orientation. While this feature is of great interest from a design standpoint, it was noted that only the fully parallel trimer depicted in the model can form in a biological system where the topology of a single-spanning TMD is constrained by the biosynthetic machinery in a type-I orientation.

It was concluded that the computational selection of oligomeric state and the sequence-diversification steps described above provide a practical approach to implement negative-design principles in TMD design; this approach is likely also be critical to de novo design hetero-oligomeric TMDs.

### Design Strategy

The results of the de novo design effort, TβL selection and structural analysis thus provided validated proMPs that could be used to program chimeric receptors to form dimers or trimers using the crystallographically confirmed proMP C2.1 (used in proCAR-2; SEQ ID No. 13) and proMP 1.2 (used in proCAR-3; SEQ ID No. 2). Also designed was a highly expressed monomeric proMP that exhibited no chloramphenicol survival in dsTβL assays, which was used to produce the monomeric proCAR-1. As a reference, a well-studied second-generation HER2-specific CAR was chosen, (using FRP5 scFv) that delivers potent cytotoxicity and cytokine release in vitro and in vivo. This CAR (herein designated Reference) incorporates the HER2-binding FRP5 scFv affinity domain, a human CD8α hinge, the human CD28 TM and costimulatory domains and the human CD3ζ-chain activation domain. A cysteine residue in the CD8α hinge mediates disulphide-bond formation that generates predominantly covalent dimers on the T cell surface. This cysteine was mutated to alanine (herein designated No Cys) to ensure that the designed TMDs were the only determinants of oligomeric state and the 23-amino-acid human CD28 TMD was replaced with monomeric, dimeric, or trimeric proMP sequences, omitting the amino-terminal proline residues. The panel of HER2 proCARs was expressed retrovirally in murine BW5147 thymoma cells, where they all reached the cell surface and were competent to signal when co-cultured with HER2+ SKBR3 human breast adenocarcinoma cells. A surface-capture and immunoblot analysis confirmed the reference CAR formed disulphide-linked dimers, while the (no) cysteine mutant and all proCARs migrated as monomers on non-reducing SDS-PAGE.

For example, a build of a proCAR may comprise an HER2-binding FRP5 scFv affinity domain, a human CD8α hinge with a Cys-to-Ala mutation, a designed TMD according to some embodiments of the present invention, a costimulatory sequence derived from CD137 (4-1BB) and/or CD28 and a human CD3ζ-chain activation domain.

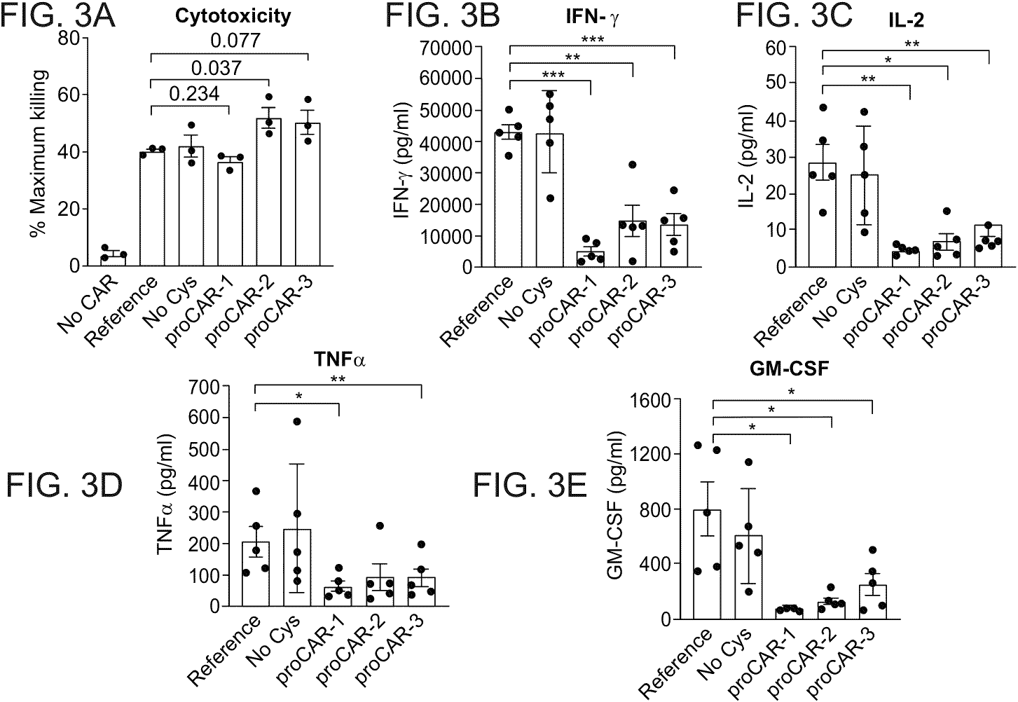

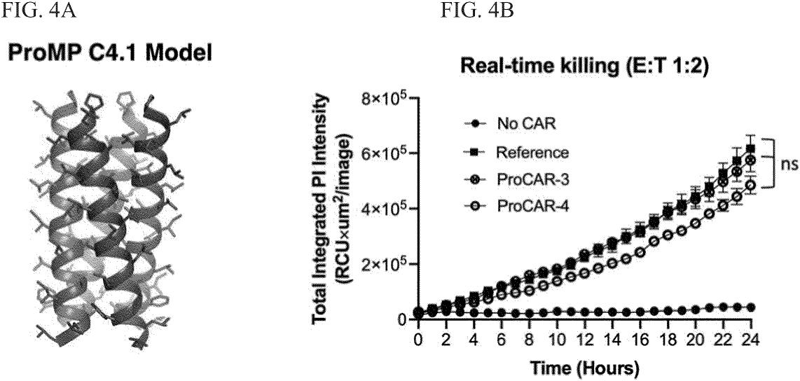

FIGS. 3A-E demonstrating that HER2 proCARs incorporating dimeric and trimeric proMP sequences provide enhanced cytotoxicity and attenuated cytokine release in vitro, wherein FIG. 3A shows a 4-hour chromium-51 release assay was performed to measure cytotoxic killing of mouse MC57-HER2 tumor cells by mouse CD8+ CAR-T cells, P values were determined using unpaired t-tests comparing Reference and proCAR-T cell data at 20:1 effector to target cell (E:T) ratio from 3 independent experiments, FIGS. 3B-E show that CAR-T cells were co-cultured with MC57-HER2 target cells for 24 hours and the indicated cytokines were measured in culture supernatants using a cytokine bead array (CBA) assay. Statistical significance (*p<0.05; **p<0.01; ***p<0.005) was determined using paired t-tests.

Retroviral transduction of purified mouse primary CD8⁺ T cells yielded 40-85% CAR⁺ T cells and similar surface expression within each experiment for all proCARs. In an in vitro cytotoxicity assay against human-HER2⁺ MC57 mouse fibrosarcoma target cells (MC57-HER2), the reference CAR and monomeric HER2 proCAR-1 yielded similar potency, while proCAR-2 and proCAR-3 produced better killers despite their lack of stabilizing intermolecular disulfide bonds. Combined data from three independent cytotoxicity experiments (FIG. 3A) showed on average approximately 25% greater cytotoxic potency for the multimeric proCAR-2 (which incorporated SEQ ID NO:13 as amino acids of positions 2-24) and proCAR-3 (which incorporated SEQ ID NO:2 as amino acids of positions 2-24), while proCAR-1 was similar to or slightly lower than the reference. Additionally, all proCAR T cells exhibited strongly attenuated IFNy, IL-2, TNFα and GM-CSF production when co-cultured with MC57-HER2 cells compared to the reference CAR, with two- to ten-fold lower levels on average across five independent experiments (FIGS. 3B-E). This effect was specific to stimulation through the engineered receptors, since all CAR-T cells secreted similar high amounts of cytokines in response to control anti-CD3/anti-CD28 bead stimulation. This independent modulation of cytotoxicity and cytokine release is best illustrated by comparing the disulphide-linked dimeric reference CAR with the non-covalently associated proCAR-2 dimer: replacing the CD28 TM domain with the proMP C2.1 (SEQ ID No. 13) sequence simultaneously conferred 25% greater cytotoxicity, 4-fold lower IFNγ and IL-2, 2.5-fold lower TNFα and 4.5-fold lower GM-CSF secretion. CAR-T cell functions were antigen- and CAR-specific since T cells transduced with an empty vector showed no cytotoxic activity and responses to antigen-negative MC57 target cells were minimal for all CARs tested.

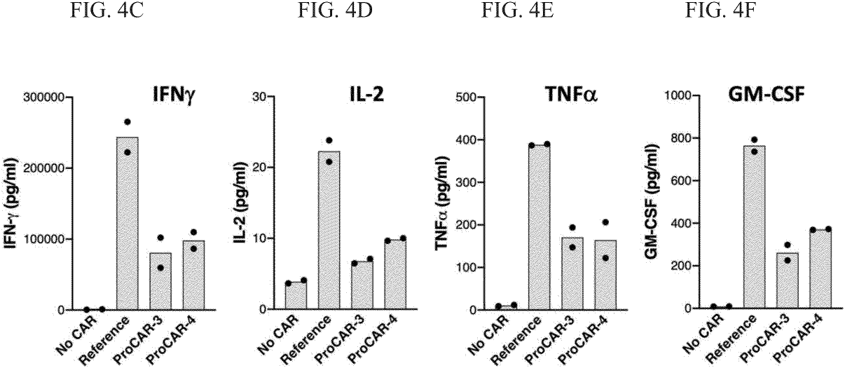

FIGS. 4A-F demonstrate that HER2 proCAR incorporating tetrameric design proMP C4.1 provide robust cytotoxicity and attenuated cytokine release in vitro, wherein FIG. 4A shows proMP C4.1 tetrameric design model, FIG. 4B shows HER2 ProCAR-4 T cells were compared to proCAR-3, Reference and No CAR T cells in a live-cell imaging (Incucyte) assay over 24 hours at low (1:2) effector to target cell (E:T) ratio, and that differences in killing curves were not significantly different in a 2-way ANOVA performed on three replicate data sets, and FIGS. 4C-F show cytokine release measured as in FIGS. 3A-E, whereas data are from one experiment with two replicate measurements.

A transmembrane sequence derived from tetrameric proMP C4.1 (SEQ ID No. 72; FIG. 4A) was incorporated to make proCAR-4, which displayed similar cytotoxic potency to the reference CAR and proCAR-3 (FIG. 4B) and still provided better than 2-fold reduction in all cytokines tested (FIGS. 4C-F).

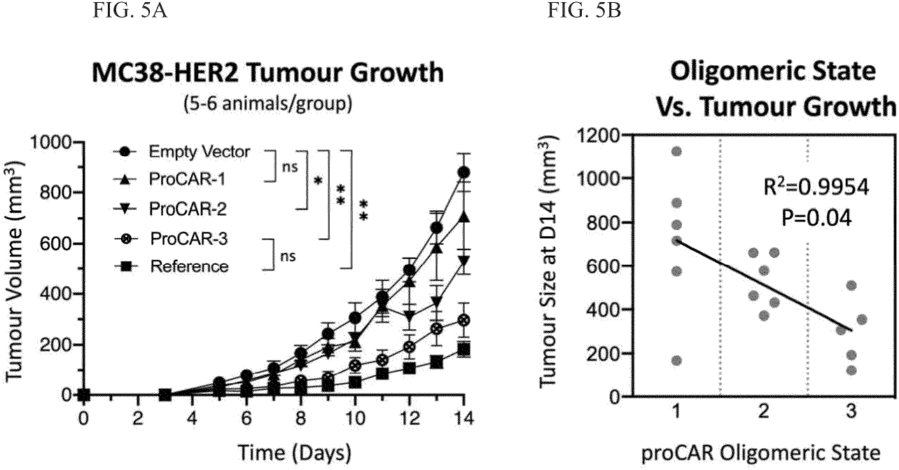

FIGS. 5A-B show that proCAR-T cells provide in vivo tumor control that scales with oligomeric state, wherein FIG. 5A shows that female NSG mice at 6-8 weeks of age (5-6 per group) were engrafted with mouse MC38-HER2 tumor cells (5×10⁵ s.c.) and 10⁷ CAR-T cells were delivered i.v. one day later (tumor growth was monitored daily using calipers) (symbols represent the mean and standard error for each group, repeated measures 2-way ANOVA test, *p<0.05, **p<0.01), and FIG. 5B presents a linear regression through the means of measurements shown in FIG. 5A show statistically significant association of tumour control with oligomeric state.

In vivo tumor control is a function of cytotoxic potency, cytokine production, proliferative capacity and CAR-T cell survival and is difficult to predict from in vitro experiments. The reference and proCARs 1-3 were therefore tested in NSG mice inoculated subcutaneously with MC38 mouse adenocarcinoma cells stably expressing HER2 and treated with 10⁷ proCAR-T cells given intravenously one day later (FIGS. 5A-B). Tumor growth in mice receiving proCAR-1 was not significantly different from the no CAR control (i.e. provided no benefit), but proCAR-2 and proCAR-3 progressively slowed tumor growth and increased survival in correlation with increasing oligomeric state. ProCAR-3 provided the closest control to the reference CAR, and with 2- to 5-fold lower inflammatory cytokine production in vitro (FIGS. 3A-E) the ability to increase CAR-T cell dosage could provide significant efficacy benefit while maintaining reduced toxicity.

### De-Novo TMD Sequences

According to some embodiments of the present invention, the method for designing novel de novo TMDs with the traits and functionality as presented hereinabove, provides a well-defined and restricted sequence space for each of the 12 de novo designed proteins (proMPs/proCARs). For each of the proMP/proCAR presented herein, the method provides optional sequences in the form of a per-position table, denoting the optional amino acids that were found suitable to occupy the given position. In essence, each of the tables presented below defines the combinatorial sequence space, wherein each sequence of the enumerated sequences that can be derived from any one of the tables below is a de novo design of the given proMP/proCAR. In the set of tables presented below the each entry is an amino acid position (number shown in the left column), the original design amino acid (first letter from the left in the second column) followed by the optional amino acid(s) that can replace the amino acid of the original design. For example, in Table 1, the fifth position is occupied by Leu in the original design, and the optional amino acids that can take its place, according to some embodiments of the present invention, are Ile, Val, Phe, Met, Ser, Thr, Trp and Tyr, in no particular order. Another example in Table 1 is the 11^{th} position, which has Ala in the original design and can be replaced only by Ser, while in position 18 the Gly in the original design is conserved throughout the sequence space of proMP 1.1 (SEQ ID No. 1), and cannot be replaced.

For a non-limiting example, proMPs that are encompassed by the scope of the present invention include, in the exemplary case of Table 1, an amino acid sequence constituting on the third amino acid from the first entry, namely I, the sixth amino acid from the second entry, namely S, the last amino acid from the third entry, namely V..., and so forth. The same per entry selection of amino acids is contemplated for all Tables 1-15 below.

TABLE 1

Sequence space for proMP 1.1 PLSFLLGILAALVGFIIGFLAALI (SEQ ID No. 1)

| Position (numbering according design model) | Sequence space (original aa first from the left) |
|---|---|
| 1 | P/L/I/V/F/M/S/T/W/Y |
| 2 | L/I/V/F/M/S/T/W/Y |
| 3 | S/A/F/G/I/L/M/T/V |
| 4 | F/A/G/I/L/M/S/T/V |
| 5 | L/I/V/F/M/S/T/W/Y |
| 6 | L/I/V/F/M/S/T/W/Y |
| 7 | G/A |
| 8 | I/L/V/F/M/S/T/W/Y |
| 9 | L/I/V/F/M/S/T/W/Y |
| 10 | A/F/L/M/S/T |
| 11 | A/S |
| 12 | L/I/V/F/M/S/T/W/Y |
| 13 | V/L/I/F/M/S/T/W/Y |
| 14 | G/A |
| 15 | L/I/V/F/M/S/T/W/Y |
| 16 | I/L/V/F/M/S/T/W/Y |
| 17 | I/L/V/F/M/S/T/W/Y |
| 18 | G |
| 19 | F/L/I/V/M/S/T/W/Y |
| 20 | L/I/V/F/M/S/T/W/Y |
| 21 | A/F/I/L/T/V |
| 22 | A/S |
| 23 | L/I/V/F/M/S/T/W/Y |
| 24 | I/L/V/F/M/S/T/W/Y |

As well as providing designed TMD sequences, Table 1 defines a proMP 1.1 (SEQ ID No. 1) motif (Motif 1) beginning at position 7 and ending at position 22 of SEQ ID No. 1, where the sequence of the motif is G/A-XXX-A/S-XX-G/A-XXX-G-XXX-A/S, wherein “X” is often, but not always, any one of L/I/V/F/M/S/T/W/Y. Such a motif, when present in a TMD sequence, comprises a part of the proMP 1.1 (SEQ ID No. 1) sequence space.

TABLE 2

Sequence space for proMP 1.2 PLLFILVAILGGLFGAIVAFLLAL (SEQ ID No. 2)

| Position (numbering according design model) | Sequence space (original aa first from the left) |
|---|---|
| 1 | P/L/I/V/F/M/S/T/W/Y |
| 2 | L/I/V/F/M/S/T/W/Y |
| 3 | L/I/V/F/M/S/T/W/Y |
| 4 | F/A/I/L/M/S/V |
| 5 | I |
| 6 | L/I/V/F/M/S/T/W/Y |
| 7 | V/A/I/L/M/T |
| 8 | A |
| 9 | I |
| 10 | L/I/V/F/M/S/T/W/Y |
| 11 | G |
| 12 | G/A/M/S |
| 13 | L/I/V/F/M/S/T/W/Y |
| 14 | F/L/I/V/M/S/T/W/Y |
| 15 | G |
| 16 | A/F/I/M/S |
| 17 | I/L/V/F/M/S/T/W/Y |
| 18 | V/L/I/F/M/S/T/W/Y |
| 19 | A/S |
| 20 | F/I/L/M |
| 21 | L/I/V/F/M/S/T/W/Y |
| 22 | L/I/V/F/M/S/T/W/Y |
| 23 | A/M/S/T/V |
| 24 | L/I/V/F/M/S/T/W/Y |

As well as providing designed TMD sequences, Table 2 defines a proMP 1.2 (SEQ ID No. 2) motif (Motif 2), beginning at position 5 and ending at position 19 of SEQ ID No. 2, where the sequence of the motif is I-XX-AI-X-G-G/A/M/S-XX-G-XXX-A/S, wherein “X” is often, but not always, any one of L/I/V/F/M/S/T/W/Y. Such a motif when present in a TMD sequence, comprises a part of the proMP 1.2 (SEQ ID No. 2) sequence space.

TABLE 3

Sequence space for proMP 1.3 PILFAILAAFIGAFIAALFVLVLA (SEQ ID No. 3)

| Position (numbering according design model) | Sequence space (original aa first from the left) |
|---|---|
| 1 | P/L/I/V/F/M/S/T/W/Y |
| 2 | I/L/V/F/M/S/T/W/Y |
| 3 | L/I/V/F/M/S/T/W/Y |
| 4 | F/A/G/I/L/M/S/T/V |
| 5 | A/I/L/M/T/V |
| 6 | I |
| 7 | L/I/V/F/M/S/T/W/Y |
| 8 | A/M/S |
| 9 | A/M/S |
| 10 | F/L/I/V/M/S/T/W/Y |
| 11 | I/L/V/F/M/S/T/W/Y |
| 12 | G/A |
| 13 | A/S |
| 14 | F/L/I/V/M/S/T/W/Y |
| 15 | I/L/V/F/M/S/T/W/Y |
| 16 | A/M/S/T |
| 17 | A/F/I/L/M/S/V |
| 18 | L/I/V/F/M/S/T/W/Y |
| 19 | F/I/L/M/V |
| 20 | V/A/F/I/L/M/T |
| 21 | L/I/V/F/M/S/T/W/Y |
| 22 | V/L/I/F/M/S/T/W/Y |
| 23 | L/I/V/F/M/S/T/W/Y |
| 24 | A/F/I/L/M/S/T/V |

As well as providing designed TMD sequences, Table 3 defines a proMP 1.3 (SEQ ID No. 3) motif (Motif 3) beginning at position 6 and ending at position 16 of SEQ ID No. 3 where the sequence of the motif is I-X-A/M/S-A/M/S-XX-G/A-A/S-XX-A/M/S/T, wherein “X” is often, but not always, any one of L/I/V/F/M/S/T/W/Y, that when present in a TMD sequence, comprises a part of the proMP 1.3 (SEQ ID No0.3) sequence space.

TABLE 4

Sequence space for proMP 1.4 PFGALLAIIAFVVGMLFSAFVLLI (SEQ ID No. 4)

| Position (numbering according design model) | Sequence space (original aa first from the left) |
|---|---|
| 1 | PL/I/V/F/M/S/T/W/Y |
| 2 | F/L/I/V/M/S/T/W/Y |
| 3 | G/A |
| 4 | A/F/I/L/M/S/T/V |
| 5 | L/I/V/F/M/S/T/W/Y |
| 6 | L/I/V/F/M/S/T/W/Y |
| 7 | A |
| 8 | I/L/V/F/M/S/T/W/Y |
| 9 | I/L/V/F/M/S/T/W/Y |
| 10 | A/M/S |
| 11 | F/L/I/V/M/S/T/W/Y |
| 12 | V/L/I/F/M/S/T/W/Y |
| 13 | V/L/I/F/M/S/T/W/Y |
| 14 | G |
| 15 | M/A/F/I/L/S/V |
| 16 | L/I/V/F/M/S/T/W/Y |
| 17 | F/L/I/V/M/S/T/W/Y |
| 18 | S/A/F/M/T |
| 19 | A/L/I/V/F/M/S/T/W/Y |
| 20 | F/L/I/V/M/S/T/W/Y |
| 21 | V/L/I/F/M/S/T/W/Y |
| 22 | L/I |
| 23 | L/I/V/F/M/S/T/W/Y |
| 24 | I/L/V/F/M/S/T/W/Y |

As well as providing designed TMD sequences, Table 4 defines a proMP 1.4 (SEQ ID No. 4) motif (Motif 4) beginning at position 7 and ending at position 22 of SEQ ID No. 4 where the sequence of the motif is G/A-XXX-A-XX-A/M/S-XXX-G-XXX-S/A/F/M/T-XXX-L/I, wherein “X” is often, but not always, any one of L/I/V/F/M/S/T/W/Y. Such a motif, when present in a TMD sequence, comprises a part of the proMP 1.4 (SEQ ID No. 4) sequence space.

TABLE 5

Sequence space for proMP 1.5 PFSLFLGVIAGIIAAFIVLFLALL (SEQ ID No. 5)

| Position (numbering according design model) | Sequence space (original aa first from the left) |
|---|---|
| 1 | P/L/I/V/F/M/S/T/W/Y |
| 2 | F/L/I/V/M/S/T/W/Y |
| 3 | S/A/F/G/I/L/M/T/V |
| 4 | L/A/I/M/T/V |
| 5 | F/L/I/V/M/S/T/W/Y |
| 6 | L/I/V |
| 7 | G/A/F/L/M/S |
| 8 | V/L/I/F/M/S/T/W/Y |
| 9 | I/L/V/F/M/S/T/W/Y |
| 10 | A/F/I/L/M/S/T/V |
| 11 | G/A/F/I/L/M/S/T/V |
| 12 | I/L/V/F/M/S/T/W/Y |
| 13 | I/L/V/F/M/S/T/W/Y |
| 14 | A/F/M/S/T |
| 15 | A/F/L/M/S |
| 16 | F/L/I/V/M/S/T/W/Y |
| 17 | I/L/V/F/M/S/T/W/Y |
| 18 | V/A/F/I/L/M/T |
| 19 | L/I/V |
| 20 | F/L/I/V/M/S/T/W/Y |
| 21 | L/I/V/F/M/S/T/W/Y |
| 22 | A/F/I/M/S/T/V |
| 23 | L/I/V/F/M/S/T/W/Y |
| 24 | L/I/V/F/M/S/T/W/Y |

As well as providing designed TMD sequences, Table 5 defines a proMP 1.5 (SEQ ID No. 5) motif (Motif 5) beginning at position 6 and ending at position 19 of SEQ ID No. 5 where the sequence of the motif is L/I/V-XXXXXXX-A/F/M/S/T-A/F/M/S/T-XXX-L/I/V, wherein “X” is often, but not always, any one of L/I/V/F/M/S/T/W/Y. Such a motif, when present in a TMD sequence, comprises a part of the proMP 1.5 (SEQ ID No. 5) sequence space.

TABLE 6

Sequence space for proMP 1.6 PFLSLVGALIGAFIAFILALFILV (SEQ ID No. 6)

| Position (numbering according design model) | Sequence space (original aa first from the left) |
|---|---|
| 1 | P/L/I/V/F/M/S/T/W/Y |
| 2 | F/L/I/V/M/S/T/W/Y |
| 3 | L/A/M/S |
| 4 | S/A/F/G/I/L/M/T/V |
| 5 | L/I/V/F/M/S/T/W/Y |
| 6 | I/L/V/F/M/S/T/W/Y |
| 7 | G/A |
| 8 | A/F/I/L/M/S/T/V |
| 9 | L/I/V/F/M/S/T/W/Y |
| 10 | I/L/V/F/M/S/T/W/Y |
| 11 | G |
| 12 | A/F/S |
| 13 | F/L/I/V/M/S/T/W/Y |
| 14 | I/L/V/F/M/S/T/W/Y |
| 15 | A |
| 16 | F/L/I/V/M/S/T/W/Y |
| 17 | I/L/V/F/M/S/T/W/Y |
| 18 | L/I/V/F/M/S/T/W/Y |
| 19 | A/T/V |
| 20 | L/I/V/F/M/S/T/W/Y |
| 21 | F/L/I/V/M/S/T/W/Y |
| 22 | I/L/V/F/M/S/T/W/Y |
| 23 | L/F/I |
| 24 | V/L/I/F/M/S/T/W/Y |

As well as providing designed TMD sequences, Table 6 defines a proMP 1.6 (SEQ ID No. 6) motif (Motif 6) beginning at position 7 and ending at position 23 of SEQ ID No. 6 where the sequence of the motif is L/A/M/S-XXX-G/A-XXX-G-A/F/S-XX-A-XXX-A/T/V-XXX-L/F/I, wherein “X” is often, but not always, any one of L/I/V/F/M/S/T/W/Y. Such a motif, when present in a TMD sequence, comprises a part of the proMP 1.6 (SEQ ID No. 6) sequence space.

TABLE 7

Sequence space for proMP 1.7 PILITLAMLTGAVIGAISSFLLVY (SEQ ID No. 7)

| Position (numbering according design model) | Sequence space (original aa first from the left) |
|---|---|
| 1 | P/L/I/V/F/M/S/T/W/Y |
| 2 | I/L/V/F/M/S/T/W/Y |
| 3 | L/I/V/F/M/S/T/W/Y |
| 4 | I/L |
| 5 | T |
| 6 | L/I/V/F/M/S/T/W/Y |
| 7 | A/F/I/L/M/S/V |
| 8 | M |
| 9 | L/I/V/F/M/S/T/W/Y |
| 10 | T |
| 11 | G/A |
| 12 | A/M/S |
| 13 | V/L/I/F/M/S/T/W/Y |
| 14 | I/L/V/F/M/S/T/W/Y |
| 15 | G |
| 16 | A/F/I/L/M/S/T/V |
| 17 | I/L/V/F/M/S/T/W/Y |
| 18 | S |
| 19 | S/A/F/I/L/M/T/V |
| 20 | F/L/I/V/M/S/T/W/Y |
| 21 | L/I/V/F/M/S/T/W/Y |
| 22 | L/I/V/F/M/S/T/W/Y |
| 23 | V/A/F/I/L/M/S/T |
| 24 | Y/L/I/V/M/S/T/W/F |

As well as providing designed TMD sequences Table 7 defines a proMP 1.7 (SEQ ID No. 7) motif (Motif 7) beginning at position 4 and ending at position 18 of SEQ ID No. 7 where the sequence of the motif is I/L-T-XX-M-X-T-G/A-A/M/S-XX-G-XX-S, wherein “X” is often, but not always, any one of L/I/V/F/M/S/T/W/Y. Such a motif, when present in a TMD sequence, comprises a part of the proMP 1.7 (SEQ ID No. 7) sequence space.

TABLE 8

Sequence space for proMP 1.8 PAFYIILAILTSFIAYLVGLLVSF (SEQ ID No. 8)

| Position (numbering according design model) | Sequence space (original aa first from the left) |
|---|---|
| 1 | P/L/I/V/F/M/S/T/W/Y |
| 2 | A |
| 3 | F/L/I/V/M/S/T/W/Y |
| 4 | A/F/L/M/S/T |
| 5 | I/L/T/V |
| 6 | I/L/V/F/M/S/T/W/Y |
| 7 | L/I |
| 8 | A/I/M/S/T/V |
| 9 | I/F/L |
| 10 | L/I/V/F/M/S/T/W/Y |
| 11 | T/A/F/G/I/L/M/S/V |
| 12 | S/A/F/G/L/M/T |
| 13 | F |
| 14 | I |
| 15 | A/S |
| 16 | A/F/I/L/M |
| 17 | L/I/V/F/M/S/T/W/Y |
| 18 | V/L/I/F/M/S/T/W/Y |
| 19 | G/A |
| 20 | L/I |
| 21 | L/I/V/F/M/S/T/W/Y |
| 22 | V/A/F/I/L/M/S/T |
| 23 | S/A/F/L/M/T/V |
| 24 | F/L/I/V/M/S/T/W/Y |

As well as providing designed TMD sequences, Table 8 defines a proMP 1.8 (SEQ ID No. 8) motif (Motif 8) beginning at position 2 and ending at position 20 of SEQ ID No. 8 where the sequence of the motif is A-XX-I/L/T/V-X-L/I-X-I/F/L-XXX-FI-A/S-XXX-G/A-L/I, wherein “X” is often, but not always, any one of L,I/V/F/M/S/T/W/Y. Such a motif, when present in a TMD sequence, comprises a part of the proMP 1.8 (SEQ ID No. 8) sequence space.

TABLE 9

Sequence space for proMP 1.9 PIYVILAILAA VFTWFTVL1TSLS (SEQ ID No. 9)

| Position (numbering according design model) | Sequence space (original aa first from the left) |
|---|---|
| 1 | P/L/I/V/F/M/S/T/W/Y |
| 2 | I/A/G/L/M/S/T/V |
| 3 | A/F/M/S |
| 4 | V/A/F/I/L/M/T |
| 5 | I/L/V/F/M/S/T/W/Y |
| 6 | L/I |
| 7 | A/F/I/L/M/S/V |
| 8 | I/L/V/F/M/S/T/W/Y |
| 9 | L/I/V/F/M/S/T/W/Y |
| 10 | A/S/T |
| 11 | A/F/I/M/S/T/V |
| 12 | V/L/I/F/M/S/T/W/Y |
| 13 | F/L/I/V/M/S/T/W/Y |
| 14 | T/A/F/I/L/M/S/V |
| 15 | A/F/I/LIM/S/TIV |
| 16 | F/L/I/V/M/S/T/W/Y |
| 17 | I/L/V/F/M/S/T/W/Y |
| 18 | V/A/F/I/L/M |
| 19 | L/I/V/F/M/S/T/W/Y |
| 20 | I/L/V/F/M/S/T/W/Y |
| 21 | T/A/F/G/I/L/M/S/V |
| 22 | S/A/F/I/L/M/T/V |
| 23 | L/I/V/F/M/S/T/W/Y |
| 24 | S/L/I/V/F/M/S/T/W/Y |

As well as providing designed TMD sequences Table 9 defines a proMP 1.9 (SEQ ID No. 9) motif (Motif 9) beginning at position 3 and ending at position 10 of SEQ ID No. 9 where the sequence of the motif is A/F/M/S-XX-L/I-XXX-A/S/T, wherein “X” is often, but not always, any one of L/I/V/F/M/S/T/W/Y. Such a motif, when present in a TMD sequence, comprises a part of the proMP 1.9 (SEQ ID No. 9) sequence space.

TABLE 10

Sequence space for proMP 1.10 PTVTSAILGVSFGTFISLVALWLA (SEQ ID No. 10)

| Position (numbering according design model) | Sequence space (original aa first from the left) |
|---|---|
| 1 | P/L/I/V/F/M/S/T/W/Y |
| 2 | T/A/I/L/M/S/V |
| 3 | V/L/I/F/M/S/T/W/Y |
| 4 | T |
| 5 | S/A/F/L/V |
| 6 | A/F/L/M/S/T |
| 7 | I/L/V/F/M/S/T/W/Y |
| 8 | L/I/V/F/M/S/T/W/Y |
| 9 | G |
| 10 | V/A/F/I/L/M/S/T |
| 11 | S/L/I/V/F/M/S/T/W/Y |
| 12 | F/L/I/V/M/S/T/W/Y |
| 13 | G |
| 14 | T/A/F/G/I/L/M/S/V |
| 15 | F/L/I/V/M/S/T/W/Y |
| 16 | I/F/L/V |
| 17 | S/A/G/L/M/T/V |
| 18 | L/I/V/F/M/S/T/W/Y |
| 19 | V/A/F/I/L/M/S |
| 20 | A |
| 21 | L/I/V/F/M/S/T/W/Y |
| 22 | W/L/I/V/F/M/S/T/Y |
| 23 | L/I/V/F/M/S/T/W/Y |
| 24 | A/G/S |

As well as providing designed TMD sequences Table 10 defines a proMP 1.10 (SEQ ID No. 10) motif (Motif 10) beginning at position 4 and ending at position 24 of SEQ ID No. 10 wherein the sequence of the motif is T-S/A/F/L/V-XXX-G-XXX-G-XX-I/F/L/V-XXX-A-XXX-A/G/S, wherein “X” is often, but not always, any one of L/I/V/F/M/S/T/W/Y. Such a motif, when present in a TMD sequence, comprises a part of the proMP 1.10 (SEQ ID No. 10) sequence space.

TABLE 11

Sequence space for proMP 1.11 PVIAILTFIVLTAISGALLAVWFS (SEQ ID No. 11)

| Position (numbering according design model) | Sequence space (original aa first from the left) |
|---|---|
| 1 | I/L/V/F/M/S/T/W/Y |
| 2 | V/L/I/F/M/S/T/W/Y |
| 3 | I/L/V/F/M/S/T/W/Y |
| 4 | A/L/1/V/F/M/S/T/W/Y |
| 5 | I/L/V/F/M/S/T/W/Y |
| 6 | L/I/V/F/M/S/T/W/Y |
| 7 | T/I/L/V/F/M/S/T/W/Y |
| 8 | F/I/L/V/M/S/T/W/Y |
| 9 | I/F/L |
| 10 | V |
| 11 | L |
| 12 | T/A/F/I/L/M/S/V |
| 13 | A/F/I/L/M/S/T/V |
| 14 | I/L/V/F/M/S/T/W/Y |
| 15 | S/I/L/V/F/M/S/T/W/Y |
| 16 | G/A |
| 17 | A/F/I/L/M/S/V |
| 18 | L/I/V/F/M/S/T/W/Y |
| 19 | L/I/V/F/M/S/T/W/Y |
| 20 | A |
| 21 | V/I/L/F/M/S/T/W/Y |
| 22 | W/V/I/L/F/M/S/T//Y |
| 23 | F/L/I/V/M/S/T/W/Y |
| 24 | S/A/F/I/L/M/T/V |

As well as providing designed TMD sequences, Table 11 defines a proMP 1.11 (SEQ ID No. 11) motif (Motif 11) beginning at position 9 and ending at position 20 of SEQ ID No. 11 where the sequence of the motif is I/F/L-VL-XXXX-G/A-XXX-A, wherein “X” is often, but not always, any one of L/I/V/F/M/S/T/W/Y. Such a motif, when present in a TMD sequence, comprises a part of the proMP 1.11 (SEQ ID No. 11) sequence space.

TABLE 12

Sequence space for proMP 1.12 PIVLLLSLLASVFGAFITFIWAYL (SEQ ID No. 12)

| Position (numbering according design model) | Sequence space (original aa first from the left) |
|---|---|
| 1 | I/L/V/F/M/S/T/W/Y |
| 2 | I/L/V/F/M/S/T/W/Y |
| 3 | V/A/F/G/I/L/M/S/T |
| 4 | L/I/V/F/M/S/T/W/Y |
| 5 | L/I/V/F/M/S/T/W/Y |
| 6 | L/I/V/F/M/S/T/W/Y |
| 7 | S/A/F/I/L/M/T/V |
| 8 | L/I/V/F/M/S/T/W/Y |
| 9 | L/I/V/F/M/S/T/W/Y |
| 10 | A/F/M/S |
| 11 | S/A/F/G/I/L/M/T/V |
| 12 | V/L/I/F/M/S/T/W/Y |
| 13 | F/L/I/V/M/S/T/W/Y |
| 14 | G/A/F/L/M/S |
| 15 | A/F/I/M/S/V |
| 16 | F/L/I/V/M/S/T/W/Y |
| 17 | I/F/L/M |
| 18 | T/A/F/I/M/S/V |
| 19 | F/L/I/V/M/S/T/W/Y |
| 20 | I/L/V/F/M/S/T/W/Y |
| 21 | W/I/L/V/F/M/S/T/Y |
| 22 | A/T/V |
| 23 | A/F/G/I/L/M/S/T/V |
| 24 | L/I/V/F/M/S/T/W/Y |

As well as providing designed TMD sequences, Table 12 defines a proMP 1.12 (SEQ ID No. 12) motif (Motif 12) beginning at position 10 and ending at position 22 of SEQ ID No. 12 where the sequence of the motif is A/F/M/S-XXX-G/A/F/L/M/S-A/F/I/M/S/V-X-I/F/L/M-XXXX-A/T/V, wherein “X” is often, but not always, any one of L/I/V/F/M/S/T/W/Y. Such a motif, when present in a TMD sequence, comprises a part of the proMP 1.12 (SEQ ID No. 12) sequence space.

TABLE 13

Sequence space for proMP C2.1 PLTVALILGIFLGTFIAFWVVYLL (SEQ ID No. 13)

| Position (numbering according design model) | Sequence space (original aa first from the left) |
|---|---|
| 1 | P/I/L/V/F/M/S/T/W/Y |
| 2 | L/I/V/F/M/S/T/W/Y |
| 3 | T/A/F/I/L/M/S/V |
| 4 | V/L/I/F/M/S/T/W/Y |
| 5 | A/F/S |
| 6 | L/I |
| 7 | I |
| 8 | L/I/V/F/M/S/T/W/Y |
| 9 | G |
| 10 | I/A/M/T/V |
| 11 | F/L/I/V/M/S/T/W/Y |
| 12 | L/I/V/F/M/S/T/W/Y |
| 13 | G |
| 14 | T/A/F/G/I/L/M/S/V |
| 15 | F/L/I/V/M/S/T/W/Y |
| 16 | I/L/V/F/M/S/T/W/Y |
| 17 | A/S/V |
| 18 | F/L/I/V/M/S/T/W/Y |
| 19 | W/F/L/I/V/M/S/T/Y |
| 20 | V/L/I/F/M/S/T/W/Y |
| 21 | V/A/F/I/L/M/S/T |
| 22 | Y/F/L/I/V/M/S/T/W |
| 23 | L/I/V/F/M/S/T/W/Y |
| 24 | L/I/V/F/M/S/T/W/Y |

As well as providing designed TMD sequences, Table 13 defines a proMP C2.1 motif (Motif 13) beginning at position 5 and ending at position 17 of SEQ ID No. 13 where the sequence of the motif A/F/S-L/I-I-X-G-I/A/M/T/V-XX-G-XXX-A/S/V, wherein “X” is often, but not always, any one of L/I/V/F/M/S/T/W/Y. Such a motif, when present in a TMD sequence, comprises a part of the proMP C2.1 (SEQ ID No. 13) sequence space.

TABLE 14

Sequence space for proMP C3.1 TALLVAFVAYYTALIALIFAILAT (SEQ ID No. 14)

| Position (numbering according design model) | Sequence space (original aa first from the left) |
|---|---|
| 1 | T/A/F/G/I/L/M/S/V |
| 2 | A/G/S |
| 3 | L/I |
| 4 | L/A/F/I/M/S/T/V/W |
| 5 | V/A/F/I/L/M/S/T/W/Y |
| 6 | A |
| 7 | F/A/I/K/L/M/R/V |
| 8 | V/A/F/I/L/M/S/T/W/Y |
| 9 | A |
| 10 | Y/A/F/I/K/L/M/S/T/V/W |
| 11 | Y/A/F/G/I/L/M/S/T/V/W |
| 12 | T/A//F/I/L/M/S/V/W/Y |
| 13 | A |
| 14 | L/I/V/ |
| 15 | I/A/F/L/M/T/V/ |
| 16 | A/G |
| 17 | L/F |
| 18 | I/F/L/M/V/W |
| 19 | F/A/I/L/M/S/T/V/W/Y |
| 20 | A |
| 21 | I/A/L/M/S/V |
| 22 | L/A/I/W |
| 23 | A/S |
| 24 | T/A/F/G/I/L/M//V/W/Y |

As well as providing designed TMD sequences, Table 14 defines a proMP C3.1 (SEQ ID No. 14) motif (Motif 14) beginning at position 2 and ending at position 23 of SEQ ID No. 14 where the sequence of the motif is A/G/S-L/I-XX-A-XX-A-XXX-A-L/I/V-X-A/G-L/F-XX-A-XX-A/S, wherein “X” is often, but not always, any one of L/I/V/F/M/S/T/W/Y. Such a motif, when present in a TMD sequence, comprises a part of the proMP C3.1 (SEQ ID No. 14) sequence space.

TABLE 15

Sequence space for proMP C4.1 PLLVALLALLAVIAALLAAIFALL (SEQ ID No. 71)

| Position (numbering according design model) | Sequence space (original aa first from the left) |
|---|---|
| 1 | P |
| 2 | L/A/F/G/H/I/M/S/T/V/W/Y |
| 3 | L/A/I/T/V |
| 4 | V/A/I/L/M/S/T |
| 5 | A/G/I/L/M/S/T/V/W |
| 6 | L/F/I/M/T/V |
| 7 | L |
| 8 | A/G/S |
| 9 | L/A/F/I/M |
| 10 | L/I/M/V |
| 11 | A/G/S |
| 12 | V/A/F/G/H/L/M/S/T/W/Y |
| 13 | I/F/L/V |
| 14 | A/F/G/L/M/S/W/Y |
| 15 | A/G/S |
| 16 | L/F/I/V |
| 17 | L/F/I |
| 18 | A/S |
| 19 | A/L/M/S/T/V |
| 20 | I/F/L/V |
| 21 | F/A/H/L/W/Y |
| 22 | A/S |
| 23 | L/A/F/M/W |
| 24 | L/A/I/M/S/W |

As well as providing designed TMD sequences, Table 15 defines a proMP C4.1 (SEQ ID No. 71) motif (Motif 15) beginning at position 7 and ending at position 18 of SEQ ID No. 71 where the sequence of the motif is L-A/G/S-XX-A/G/S-XXX-A/G/S-XX-A/S, wherein “X” is often, but not always, any one of L/I/V/F/M/S/T/W/Y. Such a motif, when present in a TMD sequence, comprises a part of the proMP C4.1 (SEQ ID No. 71) sequence space.

Thus, according to an aspect of some embodiments of the present invention, there are provided de novo designed TMD sequences, which include each and every combination of per-entry (per position) amino acid presented in each of Tables 1-15 presented above, as well as TMD sequences comprising a motif as defined (Motifs 1-15).

### Use and Applications

Natural killer cells are also contemplated in the scope of the present invention, as they can be engineered to express proteins having at least one of the de novo designed TMD provided herein. Natural killer cells, also known as NK cells or large granular lymphocytes (LGL), are a type of cytotoxic lymphocyte critical to the innate immune system. The role of NK cells is analogous to that of cytotoxic T cells in the vertebrate adaptive immune response. NK cells provide rapid responses to virus-infected cells, acting at around 3 days after infection, and respond to tumor formation. Typically, immune cells detect the major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are one of the three kinds of cells differentiated from the common lymphoid progenitor, the other two being B and T lymphocytes. [2] NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting interferon gamma. In contrast to NKT cells, NK cells do not express T-cell antigen receptors (TCR) or pan T marker CD3 or surface immunoglobulins (Ig) B cell receptors, but they usually express the surface markers CD16 (FcyRIII) and CD57 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

In addition to the knowledge that natural killer cells are effectors of innate immunity, recent research has uncovered information on both activating and inhibitory NK cell receptors which play important functional roles, including self-tolerance and the sustaining of NK cell activity. NK cells also play a role in the adaptive immune response: numerous experiments have demonstrated their ability to readily adjust to the immediate environment and formulate antigen-specific immunological memory, fundamental for responding to secondary infections with the same antigen. The role of NK cells in both the innate and adaptive immune responses is becoming increasingly important in research using NK cell activity as a potential cancer therapy. Thus, in addition to CAR T cells also provided are CAR NK cells comprising a TMD, according to embodiments of the present invention.

According to an aspect of some embodiments of the present invention, the de novo designed TMD provided herein can be used in many cell-based therapies, or in solving synthetic biology problems where membrane-protein self-association is a determinant of the signaling outcome. For example, proMPs may be used to control engineered receptor signaling in cellular immunotherapies such as CAR T cell and CAR NK cell immunotherapies to treat infectious diseases, autoimmune diseases and transplant-related conditions such as but not limited to human immunodeficiency virus (HIV), hepatitis C virus (HCV) and Epstein-Barr virus (EBV); autoimmune diseases such as type I diabetes (T1D), rheumatoid arthritis (RA) and systemic lupus erythematosus (SLE); and transplant-related conditions such as rejection and graft-versus-host disease (GVHD).

The ability to attenuate CAR-T cell cytokine release while maintaining or increasing cytotoxic potency has important implications for the safety and efficacy of cellular immunotherapies. Dangerously high levels of inflammatory cytokine production early after infusion cause the most commonly observed toxicity in CAR-T cell treatments, known as cytokine release syndrome (CRS), which is characterized by fever, hypotension, respiratory distress and multi-organ failure that can be fatal if not carefully managed. CRS and associated neurotoxicity are dose-limiting and often necessitate additional treatment with expensive cytokine blocking agents and/or immunosuppressive corticosteroids, the latter of which risks eliminating any clinical benefit from the CAR-T cell treatment itself. Two phase-I clinical trials have shown that attenuated cytokine release can lead to reduced incidence of high-grade CRS and neurotoxicity without compromising the anti-tumor potency of CD19-directed CAR-T cell therapy in B-cell lymphoma patients.

CARs consist of four main domains, namely ectodomain for specific target antigen recognition and endodomain that provides costimulatory and activation signals. These two domains are connected by hinge and transmembrane domains. The sequences of the invention disclosed herein are transmembrane domain sequences. An ectodomian is preferably a scFv recognizing a tumor antigen. A hinge region can be amino acid fragments from CD8a, CD4, CD28, IgG1, and IgG4. In some cases a CAR may be hinge-less. The transmembrane domain is a sequence of the invention. An endodomain can be a CD3 ζ intracellular domain, or can contain CD3 ζ intracellular domain along with either single co-stimulatory domain or two co-stimulatory domains like CD28 and 4-1BB. For a more detailed description of CAR T cell production, see, for example, a review by Guedan S. et al., “Engineering and Design of Chimeric Antigen Receptors” [Molecular Therapy: Methods & Clinical Development, 12, March 2019; and Alka, D. et al., Lymphocytes in Cellular Therapy: Functional Regulation of CAR T Cells, Frontiers in Immunology, 9, January 2019].

In the foregoing phase I clinical trials, reduced cytokine release was demonstrated successfully by changes to multiple domains in the CAR constructs, while the mechanisms underlying the altered functional characteristics remain unclear. Moreover, previous research showed that disarming GM-CSF alone using CRISPR inactivation or blocking antibodies significantly reduces CRS symptoms in patient-derived xenograft models. The results of the experimental work presented herein show that replacing only the CAR TMD with de novo designed proMPs, according to some embodiments of the present invention, produces CAR-T cells with intrinsically low cytokine release and high cytotoxic potency.

These results are consistent with the inventors’ guiding hypothesis that the de novo designed polypeptides, according to some embodiments of the present invention, eliminate unintended interactions with endogenous T cell proteins driven by the CD28 TMD. Importantly, it is also shown that programming the oligomeric state using high-precision self-assembling proMPs provides tunable cytotoxicity: both dimeric and trimeric proCARs delivered higher potency than the monomeric proCAR and the standard CAR with a CD28 TMD in vitro. While the trimer did not provide additional advantages over the dimer in vitro, this may be related to both forms of the receptor having saturated activation potential because a threshold number of activation motifs in the receptor oligomer has been reached. In vivo, tumor control efficacy was positively correlated with oligomeric state in the order trimer > dimer > monomer. The ability to control receptor activity using modular TMDs may be particularly important for broadly expressed solid-tumor antigens such as HER2, where modulating receptor sensitivity through the selection of variable-affinity anti-HER2 scFvs facilitated the production of CAR-T cells that were responsive to HER2-high tumor cells but not to healthy tissues expressing lower levels of the antigen (Liu et al, Cancer Research 75:3596, 2015).

The use of synthetic TMD sequences that are de novo designed to form specific oligomeric structures thus has specific benefits related to controlling receptor function. It also has additional practical benefits for its ease of incorporation into engineered receptors: unlike the complex processes of mutating and selecting variable-affinity antigen-binding domains or empirically testing combinatorial multi-domain modifications in different CAR formats, the proCAR platform has the potential to be easily implemented on the background of any existing CAR designs and combined with other modifications to extracellular or intracellular sequences.

The invention thus introduces de novo TMD design principles into the rapidly developing field of engineered receptors and highlights their usefulness in producing stable and specific membrane-spanning self-assembling fragments.

In the experimental section that follows, it is demonstrated that TMD design, selection, structural validation and incorporation into synthetic receptors for therapeutic applications is feasible and can provide specific and highly desirable functional properties. The present invention provides the path to proMP design methods and sequences that will find additional applications for controlling inter-molecular cell-surface protein interactions in a wide array of synthetic and biological systems.

T cells are genetically engineered to express chimeric antigen receptors specifically directed toward antigens on a patient’s tumor cells, then infused into the patient where they attack and kill the cancer cells. Adoptive transfer of T cells expressing CARs is a promising anti-cancer therapeutic, because CAR-modified T cells can be engineered to target virtually any tumor associated antigen. Early CAR-T cell research has focused on blood cancers, whereas the first approved treatments use CARs that target the antigen CD19, present in B-cell-derived cancers such as acute lymphoblastic leukemia (ALL) and diffuse large B-cell lymphoma (DLBCL). There are also efforts underway to engineer CARs targeting many other blood cancer antigens, including CD30 in refractory Hodgkin’s lymphoma; CD33, CD123, and FLT3 in acute myeloid leukemia (AML); and BCMA in multiple myeloma. Solid tumors have presented a more difficult target, wherein identification of good antigens has been challenging: such antigens must be highly expressed on the majority of cancer cells, but largely absent on normal tissues.

The first step in the production of CAR-T cells is the isolation of T cells from human blood. CAR-T cells may be manufactured either from the patient’s own blood, known as an autologous treatment, or from the blood of a healthy donor, known as an allogeneic treatment. The manufacturing process is the same in both cases; only the choice of initial blood donor is different. First, leukocytes are isolated using a blood cell separator in a process known as leukocyte apheresis. Peripheral blood mononuclear cells (PBMC) are then separated and collected. The products of leukocyte apheresis are then transferred to a cell-processing center. In the cell processing center, specific T cells are stimulated so that they will actively proliferate and expand to large numbers. To drive their expansion, T cells are typically treated with the cytokine interleukin 2 (IL-2) and anti-CD3 antibodies. The expanded T cells are purified and then transduced with a gene encoding the engineered CAR via a retroviral vector, typically either an integrating gammaretrovirus (RV) or a lentiviral (LV) vector. These vectors are very safe in modern times due to a partial deletion of their U3 region. The new gene editing tool CRISPR/Cas9 has recently been used instead of retroviral vectors to integrate the CAR gene into specific sites in the genome. Thereafter the patient undergoes lymphodepletion chemotherapy prior to the introduction of the engineered CAR-T cells. The depletion of the number of circulating leukocytes in the patient upregulates the number of cytokines that are produced and reduces competition for resources, which helps to promote the expansion of the engineered CAR-T cells. In the context of the present invention, the CAR-T cells are engineered using the sequences for expression of at least one of the de novo designed TMD provided herein, as these are presented in Tables 1-14 hereinabove.

Thus, according to an aspect of some embodiments of the present invention, there is provided a CAR-T cell that is engineered to express at least one of the de novo designed TMD provided herein.

According to another aspect of some embodiments of the present invention, there is provided a pharmaceutical composition that includes at least one CAR-T cell that is engineered to express at least one of the de novo designed TMD provided herein.

According to another aspect of some embodiments of the present invention, there is provided a method of treating a subject in need of, which is effected by administering to the subject a therapeutically effective amount of CAR-T cells, wherein at least some of the cells are engineered to express at least one of the de novo designed TMD provided herein.

According to another aspect of some embodiments of the present invention, there is provided a use of the de novo designed TMD provided herein, in the production of engineered proteins, cells and tissues, such as engineered CAR-T cells and CAR NK cells.

In some embodiments of the present invention, the engineered proteins, cells and tissues, such as CAR-T cells or CAR NK cells, are used in the treatment of cancer in a subject, including, but not limited to, leukemia (acute and chronic lymphocytic leukemia and acute myeloid leukemia), lymphoma (Hodgkin’s and non-Hodgkin’s lymphomas), solid tumors including renal cell carcinoma, pancreatic and pleural adenocarcinoma, glioblastoma, neuroblastoma, and gastric, thyroid, ovarian and breast cancer. Applications in other cellular immunotherapies utilizing engineered receptors to treat infectious diseases such as human immunodeficiency virus (HIV), hepatitis C virus (HCV) and Epstein-Barr virus (EBV); autoimmune diseases such as type I diabetes (T1D), rheumatoid arthritis (RA) and systemic lupus erythematosus (SLE); and transplant-related conditions such as rejection and graft-versus-host disease (GVHD).

It is expected that during the life of a patent maturing from this application many relevant de novo designed TMD will be developed and the scope of the term de novo designed TMD is intended to include all such new technologies a priori.

As used herein the term “about” refers to ± 10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrases “substantially devoid of” and/or “essentially devoid of” in the context of a certain substance, refer to a composition that is totally devoid of this substance or includes less than about 5, 1, 0.5 or 0.1 percent of the substance by total weight or volume of the composition. Alternatively, the phrases “substantially devoid of” and/or “essentially devoid of” in the context of a process, a method, a property or a characteristic, refer to a process, a composition, a structure or an article that is totally devoid of a certain process/method step, or a certain property or a certain characteristic, or a process/method wherein the certain process/method step is effected at less than about 5, 1, 0.5 or 0.1 percent compared to a given standard process/method, or property or a characteristic characterized by less than about 5, 1, 0.5 or 0.1 percent of the property or characteristic, compared to a given standard.

The term “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “process” and “method” refer to manners, means, techniques and procedures for accomplishing a given task including, but not limited to, those manners, means, techniques and procedures either known to, or readily developed from known manners, means, techniques and procedures by practitioners of the chemical, material, mechanical, computational and digital arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non-limiting fashion.

### Example 1

### Computational Methods

Command lines and RosettaScripts are available below. Rosetta is available at www(dot)rosettacommons(dot)org, using git version b210d6d5a0c21208f4f874f62-b2909f926379c0f for all Rosetta calculations.

### Membrane-Protein Energy Function

All atomistic calculations used the Rosetta ref2015_memb energy function. This energy function is based on the recent Rosetta energy function 2015 (ref2015) energetics, which is dominated by van der Waals packing, electrostatics, hydrogen bonding and water solvation, with the difference that in ref2015_memb, the solvation terms are replaced with splines that recapitulate the amino acid based lipophilicity contributions observed in the dsTβL insertion profiles. The centroid-level energy function was similarly based on ref2015 with amino acid lipophilicity preferences and a biasing potential that disfavors large interhelical crossing angles that are rarely observed in natural TMDs:

$$p e n a l t y = 1.51 \times {10}^{- 4} \times {\text{θ}}^{3} - 8.925 \times {10}^{- 3} \times \text{θ+0} \text{.187} \times \text{θ−0} \text{.532}$$

where θ is the crossing angle between the helix and the membrane normal.

### TMD De Novo Design

Three- and 9-mer backbone fragments were generated for a 24-amino acid poly valine extended chain using the Rosetta fragment picker. The Fold & Dock protocol was used in all design simulations. Briefly, depending on the type of symmetry (C2, C3 or C4), the chains were symmetrically duplicated and each move was applied identically to all chains. Moves included centroid-level fragment insertion and docking, followed by all-atom sequence optimization, and backbone, sidechain and rigid-body minimization. 50,000 independent trajectories were run and the structure models were filtered using structure and energy-based criteria (the best 1% by system energy, solvent accessible surface area more than 700 Å; shape complementarity Sc more than 0.6; ΔΔG_{binding} less than -15 R.e.u.; helicality less than 0.1 R.e.u.). Resulting models were visually inspected and selected for further computational design.

### Sequence Diversification

De novo designed sequences exhibited a high propensity of the amino acid Leu. To reduce this bias, we implemented 120 steps of Monte Carlo simulated annealing sequence design. In each step, a random single amino acid change was introduced in any position (mutations were restricted to Gly, Ala, Val, Ile, Leu, Met, Phe, Tyr, or Trp). Following relaxation, the mutant was evaluated on three criteria: ΔΔG_{binding}, system energy, and the difference between the amino acid propensities in the design versus natural TMDs using the following Equation 2 (RMSD_{sequence comp}):

$$R M S {D}_{s e q u e n c e c o m p} = \sqrt{\frac{{\sum }_{a a} {\left(f \left(a {a}^{d e s i g n}\right) - f \left(a {a}^{n a t u r a l}\right)\right)}^{2}}{L}}$$

where ƒ is the frequency of a given amino acid and L is the amino acid sequence length.

The three criteria were then transformed using the “fuzzy-logic” design sigmoidal function:

$${f}_{x} = \frac{1}{1 + {e}^{\left(x - o\right) s}}$$

where x is each of the three criteria, and o and s take the following values: for ΔΔG_{binding} 3 R.e.u. and 1 R.e.u.⁻¹, respectively, for system energy 20 R.e.u. and 0.5 R.e.u.⁻¹, respectively, and for RMSD_{sequence}_{comp} 0.05 and 50, respectively. The o thresholds on binding and system energy were computed relative to the energies of the starting model in each design. The resulting functions were then integrated into a “fuzzy-logic” optimization objective function:

$${f}_{Δ Δ G b i n d i n g} ∧ {f}_{s y s t e m e n e r g y} ∧ {f}_{R M S {D}_{s e q u e n c e c o m p}}$$

### Rosetta Site-Saturation Mutagenesis Calculations

To predict the impact of mutations on ΔΔG_{binding}, a symmetric mutation scanning was used. Briefly, following mutation, the model is symmetrically refined, including through residue packing and backbone and sidechain minimization, and the energy of the mutant is compared to that of the parental design.

### Ab Initio Structure Prediction

Designed sequences were subjected to the membrane fold & dock method essentially as described in Weinstein, J.Y. et al. [ PLoS Comput. Biol., 2019, 15, e1007318]. Structure models were filtered using structure and energy-based filters: solvent accessible surface area more than 600 Å; energy less than 0; the distance between the TMD ends along the membrane normal, TMsSpanMembrane greater than 25 Å; fractional agreement between the desired topology for each position (cytosolic, membrane, external) and the designed topology SpanTopologyMatchPos more than 0.1).

To evaluate whether the ab initio structure predictions are funneled, the Z-score was computed as follows in Equation 5:

$$Z = \frac{{E}_{l o w e s t} - \underline{E}}{S T D \left(E\right)}$$

where E_{lowest} is the lowest-energy model with an RMSD of less than 2 Å to the original design model and E represents energies of models with an RMSD > 2 Å and less than 50 R.e.u from E_{lowest}. A cutoff of Z greater than 2.5 was typically used to determine whether an energy landscape was funneled.

### Rosetta Mutational-Scanning Calculations

In order to characterise the effects of mutations on the designs’ binding energy, computational mutation scanning was conducted using the FilterScan protocol in RosettaScripts (see XMLs script below). If the difference in total energy for a mutation was more than 2.5 R.e.u., the mutation was predicted to be detrimental, otherwise it is defined as neutral/beneficial.

### Fold, Dock, and Design – Rosetta Script

<ROSETTASCRIPTS>
<SCOREFXNS>
<ScoreFunction name=“score0” weights=“%%score_func_0%%” symmetric=“1”>
<Reweight scoretype=“mp_helicality” weight=“100”/>
</ScoreFunction>
<ScoreFunction name=“score1” weights=“%%score_func_1%%” symmetric=“1”>
<Reweight scoretype=“mp_helicality” weight=“100”/>
</ScoreFunction>
<ScoreFunction name=“score2” weights=“%%score_func_2%%” symmetric=“1”>
<Reweight scoretype=“mp_helicality” weight=“100”/>
</ScoreFunction>
<ScoreFunction name=“score3” weights=“%%score_func_3%%” symmetric=“1”>
<Reweight scoretype=“mp_helicality” weight=“100”/>
</ScoreFunction>
<ScoreFunction name=“score5” weights=“%%score_func_5%%” symmetric=“1”>
<Reweight scoretype=“mp_helicality” weight=“100”/>
</ScoreFunction>
<ScoreFunction name=“beta” weights=“ref2015_memb” symmetric=“1”>
<Reweight scoretype=“mp_helicality” weight=“100”/>
</ScoreFunction>
<ScoreFunction name=“betaNotSymm” weights=“ref2015_memb” symmetric=“0”>
<Reweight scoretype=“mp_helicality” weight=“100”/>
</ScoreFunction>
<ScoreFunction name=“helicality” symmetric=“1”>
<Reweight scoretype=“mp_helicality” weight=“1”/>
</ScoreFunction>
</SCOREFXNS>
<TASKOPERATIONS>
<InitializeFromCommandline name=“init”/>
<RestrictToRepacking name=“rtr”/>
</TASKOPERATIONS>
<MOVERS>
<PyMOLMover name=“pmm” keep_history=“1”/>
<SetupForSymmetry name=“symm” definition=“%%symm_file%%”/>
<SymmetricAddMembraneMover name=“add_memb” membrane_core=“%%membrane_core%%”
steepness=“%%steepness%%”>
<Span start=“%%span_start_1%%” end=“%%span_end_1%%”
orientation=“%%span_orientation_1%%”/>
<Span start=“%%span_start_2%%” end=“%%span_end_2%%”
orientation=“%%span_orientation_2%%”/>
</SymmetricAddMembraneMover>
<MembranePositionFromTopologyMover name=“init_pos”/>
<FastRelax name=“fast_relax” scorefxn=“beta”/>
Fragment movers
<SingleFragmentMover name=“frag9” fragments=“%%frags9mers%%”
policy=“uniform”/>
<SingleFragmentMover name=“frag3” fragments=“%%frags3mers%%”
policy=“smooth”/>
Fold-and-dock specific movers
<SymFoldandDockRbTrialMover name=“rbtrial” rot_mag=“8.0” trans_mag=“3.0”
rotate_anchor_to_x=“1”/>
<SymFoldandDockRbTrialMover name=“rbtrial_smooth” rot_mag=“l.0”
trans_mag=“0.1” rotate_anchor_to_x=“1”/>
<SymFoldandDockMoveRbJumpMover name=“rbjump”/>
<SymFoldandDockSlideTrialMover name=“slidetrial”/>
Random movers
<RandomMover name=“early_stage_moveset”
movers=“frag9,rbtrial,rbjump,slidetrial” weights=“1.0,0.2,1.0,0.1” repeats=“1”/>
<RandomMover name=“final_stage_moveset”
movers=“frag3,rbtrial_smooth,rbjump,slidetrial” weights=“1.0,0.2,1.0,0.1” repeats=“1”/>
Monte Carlo Movers
<GenericMonteCarlo name=“stage1” scorefxn_name=“score0”
mover_name=“early_stage_moveset” temperature=“2.0” trials=“200” recover_low=“1”/>
<GenericMonteCarlo name=“stage2” scorefxn_name=“score1”
mover_name=“early_stage_moveset” temperature=“2.0” trials=“200” recover_low=“1”/>
<GenericMonteCarlo name=“stage3a” scorefxn_name=“score2”
mover_name=“early_stage_moveset” temperature=“2.0” trials=“20” recover_low=“1”/>
<GenericMonteCarlo name=“stage3b” scorefxn_name=“score5”
mover_name=“early_stage_moveset” temperature=“2.0” trials=“20” recover_low=“1”/>
<GenericMonteCarlo name=“stage4” scorefxn_name=“score3”
mover_name=“final_stage_moveset” temperature=“2.0” trials=“400” recover_low=“1”/>
Special stage 3 logic
<ParsedProtocol name=“stage3_cyc”>
<Add mover=“stage3a”/>
<Add mover=“stage3b”/>
</ParsedProtocol>
<LoopOver name=“stage3” mover_name=“stage3_cyc” iterations=“5” drift=“1”/>
Converts the centroid-level pose to fullatom for scoring
<SwitchResidueTypeSetMover name=“fullatom” set=“fa_standard”/>
<ExtractAsymmetricPose name=“extract_asp”/>
<!--<SymPackRotamersMover name=“soft_design” scorefxn=“beta_soft”
task_operations=“init”/>-->
<TaskAwareSymMinMover name=“hard_min” scorefxn=“beta” chi=“1” bb=“1” rb=“1”
task_operations=“init”/>
<SymPackRotamersMover name=“hard_design” scorefxn=“beta”
task_operations=“init”/>
<RotamerTrialsMinMover name=“RTmin” scorefxn=“beta”
task_operations=“init,rtr”/>
</MOVERS>
<FILTERS>
<ScoreType name=“total” scorefxn=“beta” score_type=“total_score”
confidence=“0” threshold=“0”/>
<Sasa name=“a_sasa” confidence=“0”/>
<ResidueLipophilicity name=“a_res_solv” threshold=“1000” confidence=“0”/>
<SpanTopologyMatchPose name=“a_span_topo” confidence=“0”/>
<Ddg name=“a_ddg” scorefxn=“betaNotSymm” chain_num=“2” repeats=“5”
extreme_value_removal=“true” confidence=“0”/>
<PackStat name=“a_pack” confidence=“0”/>
<BuriedUnsatHbonds2 name=“a_unsat” scorefxn=“beta” confidence=“0”/>
<ShapeComplementarity name=“a_shape” confidence=“0”/>
<TMsSpanMembrane name=“a_tms_span” confidence=“1”/>
<TMsSpanMembrane name=“a_tms_span_fa” confidence=“1” min distance=“25”/>
<HelixHelixAngle name=“a_hha_ang” angle_or_dist=“angle”
start_helix_1 = “%%span_start_1%%” end_helix_1=“%%span_end_1%%”
start_helix_2=“%%span_start_2%%” end_helix_2=“%%span_end_2%%” confidence=“0”/>
<HelixHelixAngle name=“a_hha_dst_vec” angle_or_dist=“dist” dist_by_atom=“0”
start_helix_1 = “%%span_start_1%%” end_helix_1=“%%span_end_1%%”
start_helix_2=“%%span_start_2%%” end_helix_2=“%%span_end_2%%” confidence=“0”/>
<HelixHelixAngle name=“a_hha_dst_atm” angle_or_dist=“dist” dist_by_atom=“1”
start_helix_1 = “%%span_start_1%%” end_helix_1=“%%span_end_1%%”
start_helix_2=“%%span_start_2%%” end_helix_2=“%%span_end_2%%” confidence=“0”/>
<ScoreType name=“a_helicality” scorefxn=“helicality”
score_type=“total_score” confidence=“1” threshold=“3”/>
<TMsAAComp name=“a_tms_aa_comp” confidence=“0” threshold=“0”/>
</FILTERS>
<PROTOCOLS>
<Add mover=“symm”/>
<Add mover=“add_memb”/>
<Add mover=“stage1”/>
<Add mover=“stage2”/>
<Add mover=“stage3”/>
<Add mover=“stage4”/>
<Add filter=“a_helicality”/>
<Add mover=“fullatom”/>
<Add filter=“a_tms_span_fa”/>
!!!! design !!!!
<Add mover=“hard_design”/>
<Add mover=“hard_min”/>
<Add mover=“hard_design”/>
<Add mover=“hard_min”/>
<Add mover=“RTmin”/>
<Add mover=“RTmin”/>
<Add filter=“a_tms_span”/>
<Add mover=“fast_relax”/>
<Add mover=“pmm”/> # DO I WANT THIS HERE?
<Add filter=“total”/>
<Add filter=“a_sasa”/>
<Add filter=“a_res_solv”/>
<Add filter=“a_span_topo”/>
<Add filter=“a_helicality”/>
# important to remove Symmetry so RMSD can be calculated
<Add mover=“extract_asp”/>
<Add filter=“a_res_solv”/>
<Add filter=“a_pack”/>
<Add filter=“a_unsat”/>
<Add filter=“a_shape”/>
<Add filter=“a_ddg”/>
<Add filter=“a_hha_ang”/>
<Add filter=“a_hha_dst_vec”/>
<Add filter=“a_hha_dst_atm”/>
<Add filter=“a_tms_aa_comp”/>
</PROTOCOLS>
<OUTPUT scorefxn=“betaNotSymm”/>
</ROSETTASCRIPTS>

### Fold, Dock, and Design – Command Line

~/Rosetta/main/source/bin/rosetta_scripts.default.linuxgccreleas -database
Rosetta/main/database @flags_fdd
flags_fdd:
# general data
-parser:protocol fold_dock_design.xml
-in:file:fasta ploy_V.fasta
-in:file:native 24.pdb
-overwrite
-use_input_sc
-nstruct 12
-jd2:ntrials 10
#-mute all
# fragment stuff
-parser:script_vars frags9mers=frags_9ploy_V_pdbtm.200.9mers
-parser:script_vars frags3mers=frags_3ploy_V_pdbtm.200.3mers
-parser:script_vars symm_file=C2.symm
# membrane spans:
-parser:script_vars span_starts=1A
-parser:script_vars span_ends=24A
-parser:script_vars span_oris=out2in
-parser:script_vars span_start_1=1
-parser:script_vars span_end_1=24
-parser:script_vars span_orientation_1=out2in
-parser:script_vars span_start_2=25
-parser:script_vars span_end_2=48
-parser:script_vars span_orientation_2=out2in
-parser:script_vars span_starts=1A
-parser:script_vars span_ends=24A
-parser:script_vars span_oris=out2in
-parser:script_vars score_func_0=score0_memb
-parser:script_vars score_func_1=score1_memb
-parser:script_vars score_func_2=score2_memb
-parser:script_vars score_func_3=score3_memb
-parser:script_vars score_func_5=score5_memb
# energy function stuff:
-parser:script_vars energy_function=ref
# membrane adjustments
-mp:scoring:hbond
-parser:script_vars steepness=4
-parser:script_vars membrane_core=10

### Sequence Diversification – Rosetta Script

<ROSETTASCRIPTS>
<SCOREFXNS>
<ScoreFunction name=“ref” weights=“ref2015_memb” symmetric=“1”>
<Reweight scoretype=“fa_mpenv_smooth” weight=“0.000001”/>
<Reweight scoretype=“coordinate_constraint” weight=“0.4”/>
</ScoreFunction>
<ScoreFunction name=“ref_no_cst” weights=“ref2015_memb” symmetric=“1”>
<Reweight scoretype=“fa_mpenv_smooth” weight=“0.000001”/>
</ScoreFunction>
<ScoreFunction name=“helicality” symmetric=“1”>
<Reweight scoretype=“mp_helicality” weight=“1”/>
</ScoreFunction>
</SCOREFXNS>
<RESIDUE_SELECTORS>
<ResidueName name=“gly” residue_name3=“GLY”/>
<ResidueName name=“asn_gln” residue_name3=“ASN,GLN”/>
<Index name=“all_aas” resnums=“1-48”/>
<Not name=“not_aas” selector=“all_aas”/>
</RESIDUE_SELECTORS>
<TASKOPERATIONS>
<OperateOnResidueSubset name=“keep_gly” selector=“gly”>
<RestrictToRepackingRLT/>
</OperateOnResidueSubset>
<OperateOnResidueSubset name=“keep_qn” selector=“asn_gln”>
<RestrictToRepackingRLT/>
</OperateOnResidueSubset>
<OperateOnResidueSubset name=“freeze_not_aas” selector=“not_aas”>
<PreventRepackingRLT/>
</OperateOnResidueSubset>
<RestrictToRepacking name=“rtr”/>
<RestrictAbsentCanonicalAAS name=“to_restrict_avlimfwyst” resnum=“0”
keep_aas=“GAVIFSTYW”/>
<InitializeFromCommandline name=“init”/>
</TASKOPERATIONS>
<FILTERS>
<ResidueLipophilicity name=“a_res_lipo” confidence=“0”/>
<ScoreType name=“a_total” scorefxn=“ref” score_type=“total_score”
confidence=“0” threshold=“0”/>
<ScoreType name=“a_total_final” scorefxn=“ref” score_type=“total_score”
confidence=“1” threshold=“-140”/>
<Sasa name=“a_sasa” confidence=“0”/>
<SpanTopologyMatchPose name=“a_span_topo” confidence=“0”/>
<Ddg name=“a_ddg” scorefxn=“ref_no_cst” confidence=“0” />
<PackStat name=“a_pack” confidence=“1” threshold=“0.3”/>
<BuriedUnsatHbonds2 name=“a_unsat” scorefxn=“ref” confidence=“0”/>
<ShapeComplementarity name=“a_shape” confidence=“0”/>
<TMsSpanMembrane name=“a_tms_span” confidence=“0”/>
<TMsSpanMembrane name=“a_tms_span_fa” confidence=“0” min_distance=“25”/>
<HelixHelixAngle name=“a_hha_ang” angle_or_dist=“angle”
start_helix_1=“%%s1%%” end_helix_1=“%%e1%%” start_helix_2=“%%s2%%” end_helix_2=“%%e2%%”
confidence=“0”/>
<MembAccesResidueLipophilicity name=“a_marl” confidence=“0” verbose=“0”/>
<TMsAAComp name=“a_tms_aa_comp” confidence=“0” threshold=“0”/>
<BindingStrain name=“a_bind” scorefxn=“ref_no_cst” jump=“1” confidence=“1”
threshold=“5”/>
<ScoreType name=“a_helicality” scorefxn=“helicality”
score_type=“total_score” confidence=“1” threshold=“3”/>
<Sigmoid name=“a_total_sig” filter=“a_total” steepness=“0.5” offset=“20”
negate=“0” confidence=“0”/>
<Sigmoid name=“a_ddg_sig_10” filter=“a_ddg” steepness=“1” offset=“10”
negate=“0” confidence=“0”/>
<Sigmoid name=“a_ddg_sig_2” filter=“a_ddg” steepness=“1” offset=“3”
negate=“0” confidence=“0”/>
<Sigmoid name=“a_comp_sig” filter=“a_tms_aa_comp” steepness=“50”
offset=“0.05” negate=“0” confidence=“0”/>
<Operator name=“a_obj_func_ddg_10”
filters=“a_total_sig,a_ddg_sig_10,a_comp_sig” operation=“PRODUCT” logarithm=“1”
threshold=“100000” confidence=“0” negate=“1”/>
<Operator name=“a_obj_func_ddg_2”
filters=“a_total_sig,a_ddg_sig_2,a_comp_sig” operation=“PRODUCT” logarithm=“1”
threshold=“100000” confidence=“0” negate=“1”/>
</FILTERS>
<MOVERS>
<SymmetricAddMembraneMover name=“add_memb” membrane_core=“%%memb_core%%”
steepness=“%%steepness%%”>
<Span start=“%%s1%%” end=“%%e1%%” orientation=“%%o1%%”/>
<Span start=“%%s2%%” end=“%%e2%%” orientation=“%%o2%%”/>
</SymmetricAddMembraneMover>
<RandomMutation name=“mutate”
task_operations=“freeze_not_aas,init,to_restrict_avlimfwyst,keep_gly,keep_qn”
scorefxn=“ref”/>
<VirtualRoot name=“virt_root”/>
<AtomCoordinateCstMover name=“atom_cst” coord_dev=“0.5” bounded=“false”
native=“false”/>
<TaskAwareSymMinMover name=“min_mover” scorefxn=“ref” chi=“1” bb=“1” rb=“1”
task_operations=“init,rtr”/>
<ParsedProtocol name=“mutate_min”>
<Add mover=“mutate”/>
<Add mover=“min_mover”/>
</ParsedProtocol>
<GenericMonteCarlo name=“gmc_sigs_1” filter_name=“a_obj_func_ddg_10”
preapply=“0” mover_name=“mutate_min” temperature=“0.1” trials=“240” recover_low=“1”
reset_baselines=“1”/>
<GenericMonteCarlo name=“gmc_sigs_2” filter_name=“a_obj_func_ddg_2”
preapply=“0” mover_name=“mutate_min” temperature=“0.1” trials=“80” recover_low=“1”
reset_baselines=“1”/>
<GenericSimulatedAnnealer name=“sim_anneal” mover_name=“mutate_min”
filter_name=“a_obj_func_ddg_2” trials=“120” sample_type=“low” recover_low=“1”
preapply=“0” reset_baselines=“1” history=“10”/>
<SetupForSymmetry name=“symm” definition=“%%symm_file%%”
preserve_datacache=“false”/>
</MOVERS>
<PROTOCOLS>
<Add mover=“symm”/>
<Add mover=“add_memb”/>
<Add mover=“atom_cst”/>
<Add mover=“min_mover”/>
<Add mover=“sim_anneal”/>
<Add mover=“min_mover”/>
<Add filter=“a_total_sig”/>
<Add filter=“a_total_final”/>
<Add filter=“a_ddg_sig_2”/>
<Add filter=“a_ddg_sig_10”/>
<Add filter=“a_comp_sig”/>
<Add filter=“a_obj_func_ddg_2”/>
<Add filter=“a_obj_func_ddg_10”/>
<Add filter=“a_tms_aa_comp”/>
<Add filter=“a_res_lipo”/>
<Add filter=“a_marl”/>
<Add filter=“a_sasa”/>
<Add filter=“a_total”/>
<Add filter=“a_ddg”/>
<Add filter=“a_bind”/>
<Add filter=“a_pack”/>
<Add filter=“a_unsat”/>
<Add filter=“a_shape”/>
<Add filter=“a_tms_span”/>
<Add filter=“a_helicality”/>
<Add filter=“a_tms_aa_comp”/>
</PROTOCOLS>
<OUTPUT scorefxn=“ref”/>
</ROSETTASCRIPTS>

### Sequence Diversification – Command Line

~/Rosetta/main/source/bin/rosetta_scripts.default.linuxgccreleas -database
Rosetta/main/database @flags_seq_divers
flags_seq_divers:
-parser:protocol GMC_seq_diversifier.xml
-overwrite
-parser:script_vars memb_core=10
-parser:script_vars steepness=4
-mute all
-nstruct 10
-parser:script_vars s1=1
-parser:script_vars s2=24
-parser:script_vars e1=25
-parser:script_vars e2=48
-parser:script_vars o1=out2in
-parser:script_vars o2=out2in
-mp:scoring:hbond
-jd2:ntrials 1000000
-use_input_sc
-s seed_INPUT.pdb
-parser:script_vars symm_file=seed.symm

### FilterScan – Rosetta Script

<ROSETTASCRIPTS>
<SCOREFXNS>
<ScoreFunction name=“ddg_sfx” weights=“%%scorefxn%%” symmetric=“1”/>
<ScoreFunction name=“full” weights=“ref2015_memb” symmetric=“1”>
<Reweight scoretype=“coordinate_constraint” weight=“%%cst_value%%”/>
</ScoreFunction>
<ScoreFunction name=“soft” weights=“ref2015_soft” symmetric=“1”>
<Reweight scoretype=“mp_res_lipo” weight=“1”/>
<Reweight scoretype=“coordinate_constraint” weight=“%%cst_value%%”/>
</ScoreFunction>
</SCOREFXNS>
<TASKOPERATIONS>
<InitializeFromCommandline name=“init”/>
<RestrictToRepacking name=“rtr”/>
<DesignAround name=“des_around” design_shell=“0.1”
resnums=“%%current_res%%” repack_shell=“8.0”/>
<OperateOnResidueSubset name=“restrict_res”>
<Index resnums=“%%res_to_restrict%%”/>
<RestrictToRepackingRLT/>
</OperateOnResidueSubset>
<OperateOnResidueSubset name=“fix_res”>
<Index resnums=“%%res_to_fix%%”/>
<PreventRepackingRLT/>
</OperateOnResidueSubset>
</TASKOPERATIONS>
<MOVERS>
<SetupForSymmetry name=“symm” definition=“%%symm_file%%”/>
<SymmetricAddMembraneMover name=“add_memb” membrane_core=“10”
steepness=“4”>
<Span start=“1” end=“24” orientation=“in2out”/>
<Span start=“25” end=“48” orientation=“in2out”/>
</SymmetricAddMembraneMover>
<TransformIntoMembraneMover name=“tramsform” />
<AtomCoordinateCstMover name=“atom_cst” coord_dev=“0.5” bounded=“false”
native=“false”/>
<MinMover name=“min_all” scorefxn=“ddg_sfx” chi=“1” bb=“1”
jump=“%%jump%%”/>#scorefxn_full
<SymPackRotamersMover name=“soft_repack” scorefxn=“soft”
task_operations=“init,rtr”/>
<SymPackRotamersMover name=“hard_repack” scorefxn=“full”
task_operations=“init,rtr”/>
<SymRotamerTrialsMover name=“RTmin” scorefxn=“full”
task_operations=“init,rtr”/>
<SymMinMover name=“soft_min” scorefxn=“soft” chi=“1” bb=“1” jump=“0”/>
<SymMinMover name=“hard_min” scorefxn=“full” chi=“1” bb=“1” jump=“0”/>
<ParsedProtocol name=“refinement_block”> #10 movers
<Add mover_name=“soft_repack”/>
<Add mover_name=“soft_min”/>
<Add mover_name=“soft_repack”/>
<Add mover_name=“hard_min”/>
<Add mover_name=“hard_repack”/>
<Add mover_name=“hard_min”/>
<Add mover_name=“hard_repack”/>
Add mover_name=“RTmin”/>
Add mover_name=“RTmin”/>
<Add mover_name=“hard_min”/>
</ParsedProtocol>
<LoopOver name=“iter4” mover_name=“refinement_block” iterations=“4”/>
</MOVERS>
<FILTERS>
<Ddg name=“ddg” scorefxn=“ddg_sfx” threshold=“0”
repeats=“5”/>#chain_num=“2”
<ScoreType name=“stability_score_full” scorefxn=“full”
score_type=“total_score” threshold=“0.0”/>
<Delta name=“delta_score_full” filter=“stability_score_full” upper=“1”
lower=“0” range=“0.5”/> #upper and lower are booleans. Delta filters out all the
mutations that are worse or better by less than -0.55R.E.U
Delta name=“delta_score_full” filter=“stability_score_full” upper=“1”
lower=“0” range=“0.5”/> #upper and lower are booleans. Delta filters out all the
mutations that are worse or better by less than -0.55R.E.U
FilterScan name=“filter_scan” scorefxn=“ddg_sfx”
relax_mover=“min_all” keep_native=“%%keep_n%%”
task_operations=“init,des_around,fix_res,restrict_res”
delta_filters=“delta_score_full” delta=“true”
resfile_name=“%%resfiles_path%%/res_%%current_res%%” report_all=“1”
delta_filter_thresholds=“%%fs_thresholds%%”
score_log_file=“%%scores_path%%/res%%current_res%%_score_full.log” dump_pdb=“1” />
<FilterScan name=“filter_scan” scorefxn=“full” relax_mover=“iter4”
keep_native=“%%keep_n%%” task_operations=“init,des_around,fix_res,restrict_res”
delta_filters=“delta_score_full” delta=“true”
resfile_name=“%%resfiles_path%%/res_%%current_res%%” report_all=“1”
delta_filter_thresholds=“%%fs_thresholds%%”
score_log_file=“%%scores_path%%/res%%current_res%%_score_full.log” dump_pdb=“0” />
</FILTERS>
<PROTOCOLS>
<Add mover=“symm”/>
<Add mover=“add_memb”/>
Add mover=“tramsform”/>
<Add mover=“atom_cst”/>
<Add filter=“filter_scan”/>
</PROTOCOLS>
</ROSETTASCRIPTS>

### FilterScan – Command Line

~/Rosetta/main/source/bin/rosetta_scripts.default.linuxgccreleas -database
Rosetta/main/database @flags_filterscan
Flags_filterscan:
# general data
-parser:protocol filterscan_auto_refine_SYMM.xml
#-database #path to database
-overwrite
# membrane spans: #changed to in2out 23Feb17 for experimental reasons
-parser:script_vars res_to_fix=1A
-parser:script_vars res_to_restrict=1A
-parser:script_vars cst_value=0.4
-parser:script_vars jump=1
-parser:script_vars scorefxn=ref2015_memb
-parser:script_vars span_orientation_2=in2out
-parser:script_vars
fs_thresholds=0.0,0.5,1.0,1.5,2.0,2.5,3.0,3.5,4.0,4.5,5.0,5.5,6.0,6.5,7.0,8.0,9.0,10.0
,11.0,12.0
-parser:script_vars keep_n=1
#-score: :elec_memb_sig_die
#-corrections: :beta_nov16
#-score:memb_fa_sol
-mp:scoring:hbond
-use_input_sc
-parser:script_vars current_res=3
-parser:script_vars pdb_dump=#filterscan/pdbs/3_
-out:path:score score
-out:path:pdb pdbs
-parser:script_vars resfiles_path=./
-parser:script_vars scores_path=./
-parser:script_vars symm_file=seed.symm

### Ab-Initio Structure Prediction - Rosetta Script

<ROSETTASCRIPTS>
<TASKOPERATIONS>
<InitializeFromCommandline name=“init”/>
<RestrictToRepacking name=“rtr”/>
</TASKOPERATIONS>
<SCOREFXNS>
<ScoreFunction name=“score0” weights=“%%score_func_0%%” symmetric=“1”>
<Reweight scoretype=“mp_helicality” weight=“0”/>
<Reweight scoretype=“mp_span_ang” weight=“0”/>
</ScoreFunction>
<ScoreFunction name=“score1” weights=“%%score_func_1%%” symmetric=“1”>
<Reweight scoretype=“mp_helicality” weight=“0”/>
<Reweight scoretype=“mp_span_ang” weight=“0”/>
<Reweight scoretype=“mp_nonhelix” weight=“0”/>
</ScoreFunction>
<ScoreFunction name=“score2” weights=“%%score_func_2%%” symmetric=“1”>
<Reweight scoretype=“mp_helicality” weight=“0”/>
<Reweight scoretype=“mp_span_ang” weight=“0”/>
<Reweight scoretype=“mp_nonhelix” weight=“0”/>
</ScoreFunction>
<ScoreFunction name=“score3” weights=“%%score_func_3%%” symmetric=“1”>
<Reweight scoretype=“mp_helicality” weight=“0”/>
<Reweight scoretype=“mp_span_ang” weight=“0”/>
<Reweight scoretype=“mp_nonhelix” weight=“0”/>
</ScoreFunction>
<ScoreFunction name=“score5” weights=“%%score_func_5%%” symmetric=“1”>
<Reweight scoretype=“mp_helicality” weight=“0”/>
<Reweight scoretype=“mp_span_ang” weight=“0”/>
<Reweight scoretype=“mp_nonhelix” weight=“0”/>
</ScoreFunction>
<ScoreFunction name=“ref” weights=“ref2015_memb” symmetric=“1”>
<Reweight scoretype=“mp_helicality” weight=“0”/>
<Reweight scoretype=“mp_span_ang” weight=“0”/>
</ScoreFunction>
<ScoreFunction name=“refNotSymm” weights=“ref2015_memb” symmetric=“0”>
<Reweight scoretype=“mp_helicality” weight=“0”/>
<Reweight scoretype=“mp_span_ang” weight=“0”/>
</ScoreFunction>
<ScoreFunction name=“helicality” symmetric=“1”>
<Reweight scoretype=“mp_helicality” weight=“0”/>
<Reweight scoretype=“mp_span_ang” weight=“0”/>
</ScoreFunction>
<ScoreFunction name=“helicality_notsymm” symmetric=“0”>
<Reweight scoretype=“mp_helicality” weight=“0”/>
<Reweight scoretype=“mp_span_ang” weight=“0”/>
</ScoreFunction>
</SCOREFXNS>
<MOVERS>
<SetupForSymmetry name=“symm” definition=“%%symm_file%%”/>
<SymmetricAddMembraneMover name=“add_memb” membrane_core=“%%membrane_core%%”
steepness=“%%steepness%%” span_starts_num=“%%span_starts%%”
span_ends_num=“%%span_ends%%” span_orientations=“%%span_oris%%”/>
<MembranePositionFromTopologyMover name=“init_pos”/>
<FastRelax name=“fast_relax” scorefxn=“%%energy_function%%”
task_operations=“init”/>
Fragment movers
<SingleFragmentMover name=“frag9” fragments=“%%frags9mers%%”
policy=“uniform”>
<MoveMap>
<Span begin=“1” end=“24” chi=“1” bb=“1”/>
</MoveMap>
</SingleFragmentMover>
<SingleFragmentMover name=“frag3” fragments=“%%frags3mers%%”
policy=“smooth”>
<MoveMap>
<Span begin=“1” end=“24” chi=“1” bb=“1”/>
</MoveMap>
</SingleFragmentMover>
Fold-and-dock specific movers
<SymFoldandDockRbTrialMover name=“rbtrial” rot_mag=“8.0” trans_mag=“3.0”
rotate_anchor_to_x=“1”/>
<SymFoldandDockRbTrialMover name=“rbtrial_smooth” rot_mag=“1.0”
trans_mag=“0.1” rotate_anchor_to_x=“1”/>
<SymFoldandDockMoveRbJumpMover name=“rbjump”/>
<SymFoldandDockSlideTrialMover name=“slidetrial”/>
Random movers
<RandomMover name=“early_stage_moveset”
movers=“frag9,rbtrial,rbjump,slidetrial” weights=“1.0,0.2,1.0,0.1” repeats=“1”/>
<RandomMover name=“final_stage_moveset”
movers=“frag3,rbtrial_smooth,rbjump,slidetrial” weights=“1.0,0.2,1.0,0.1” repeats=“1”/>
Monte Carlo Movers
<GenericMonteCarlo name=“stage1” scorefxn_name=“score0”
mover_name=“early_stage_moveset” temperature=“2.0” trials=“200” recover_low=“1”/>
<GenericMonteCarlo name=“stage2” scorefxn_name=“score1”
mover_name=“early_stage_moveset” temperature=“2.0” trials=“200” recover_low=“1”/>
<GenericMonteCarlo name=“stage3a” scorefxn_name=“score2”
mover_name=“early_stage_moveset” temperature=“2.0” trials=“20” recover_low=“1”/>
<GenericMonteCarlo name=“stage3b” scorefxn_name=“score5”
mover_name=“early_stage_moveset” temperature=“2.0” trials=“20” recover_low=“1”/>
<GenericMonteCarlo name=“stage4” scorefxn_name=“score3”
mover_name=“final_stage_moveset” temperature=“2.0” trials=“400” recover_low=“1”/>
Special stage 3 logic
<ParsedProtocol name=“stage3_cyc”>
<Add mover=“stage3a”/>
<Add mover=“stage3b”/>
</ParsedProtocol>
<LoopOver name=“stage3” mover_name=“stage3_cyc” iterations=“5” drift=“1”/>
Converts the centroid-level pose to fullatom for scoring
<SwitchResidueTypeSetMover name=“fullatom” set=“fa_standard”/>
<ExtractAsymmetricPose name=“extract_asp” clear_sym_def=“1”/>
<MinMover name=“min_mover” scorefxn=“refNotSymm” chi=“1” bb=“1” jump=“1”/>
<PackRotamersMover name=“pack” scorefxn=“refNotSymm”
task_operations=“init,rtr”/>
<RotamerTrialsMinMover name=“RTmin” scorefxn=“refNotSymm”
task_operations=“init,rtr”/>
<DumpPdb name=“dump_pdb” fname=“dump.pdb” scorefxn=“%%energy_function%%”/>
<SwitchChainOrder name=“switch” chain_order=“123”/>
<DeleteChain name=“delete_mem” chain=“4” />
</MOVERS>
<FILTERS>
<ScoreType name=“total” scorefxn=“%%energy_function%%”
score_type=“total_score” confidence=“1” threshold=“0”/>
<Sasa name=“a_sasa” confidence=“0” threshold=“300”/>
<ResidueLipophilicity name=“a_res_lipo” threshold=“1000” confidence=“0”/>
<SpanTopologyMatchPose name=“a_span_topo” confidence=“0”/>
<Ddg name=“a_ddg” scorefxn=“%%energy_function%%NotSymm” chain_num=“2”
repeats=“5” extreme_value_removal=“true” confidence=“0” threshold=“-5”/>
<PackStat name=“a_pack” confidence=“0” threshold=“0.3”/>
<BuriedUnsatHbonds2 name=“a_unsat” scorefxn=“%%energy_function%%”
confidence=“0”/>
<ShapeComplementarity name=“a_shape” confidence=“0”/>
<TMsSpanMembrane name=“a_tms_span” confidence=“1” min_distance=“25”/>
<TMsSpanMembrane name=“a_tms_span_fa” confidence=“0” min_distance=“25”/>
<HelixHelixAngle name=“a_hha_ang” angle_or_dist=“angle”
start_helix_1=“%%span_start_1%%” end_helix_1=“%%span_end_1%%”
start_helix_2=“%%span_start_2%%” end_helix_2=“%%span_end_2%%” confidence=“0”/>
<HelixHelixAngle name=“a_hha_dst_vec” angle_or_dist=“dist” dist_by_atom=“0”
start_helix_1=“%%span_start_1%%” end_helix_1=“%%span_end_1%%”
start_helix_2=“%%span_start_2%%” end_helix_2=“%%span_end_2%%” confidence=“0”/>
<HelixHelixAngle name=“a_hha_dst_atm” angle_or_dist=“dist” dist_by_atom=“1”
start_helix_1=“%%span_start_1%%” end_helix_1=“%%span_end_1%%”
start_helix_2=“%%span_start_2%%” end_helix_2=“%%span_end_2%%” confidence=“0”/>
<MembAccesResidueLipophilicity name=“a_marl” confidence=“0” verbose=“0”/>
<ScoreType name=“a_helicality” scorefxn=“helicality_notsymm”
score_type=“mp_helicality” confidence=“0” threshold=“10”/>
<ScoreType name=“a_helicality_symm” scorefxn=“helicality”
score_type=“mp_helicality” confidence=“0” threshold=“10”/>
<MPSpanAngle name=“a_angle_1” tm=“1” ang_min=“0” ang_max=“50”
confidence=“0”/>
<MPSpanAngle name=“a_angle_2” tm=“2” ang_min=“0” ang_max=“50”
confidence=“0”/>
<BindingStrain name=“a_bind” scorefxn=“%%energy_function%%” jump=“1”
confidence=“0” threshold=“5”/>
<PoseInfo name=“info”/>
</FILTERS>
<PROTOCOLS>
<Add mover=“symm”/>
<Add mover=“add_memb”/>
<Add mover=“stage1”/>
<Add mover=“stage2”/>
<Add mover=“stage3”/>
<Add mover=“stage4”/>
<Add filter=“a_helicality_symm”/>
<Add filter=“a_angle_1”/>
<Add filter=“a_angle_2”/>
<Add mover=“fullatom”/>
<Add filter=“a_tms_span”/>
<Add mover=“fast_relax”/>
<Add filter=“total”/>
<Add filter=“a_sasa”/>
<Add filter=“a_span_topo”/>
<Add mover=“extract_asp”/>
<Add mover=“pack”/>
<Add mover=“min_mover”/>
<Add mover=“RTmin”/>
<Add mover=“RTmin”/>
<Add filter=“a_tms_span”/>
<Add filter=“total”/>
<Add filter=“a_sasa”/>
<Add filter=“a_span_topo”/>
<Add filter=“a_res_lipo”/>
<Add filter=“a_pack”/>
<Add filter=“a_unsat”/>
<Add filter=“a_shape”/>
<Add filter=“a_ddg”/>
<Add filter=“a_hha_ang”/>
<Add filter=“a_hha_dst_vec”/>
<Add filter=“a_hha_dst_atm”/>
<Add filter=“a_marl”/>
<Add filter=“a_tms_span_fa”/>
<Add filter=“a_helicality”/>
<Add filter=“a_angle_1”/>
<Add filter=“a_angle_2”/>
<Add filter=“a_bind”/>
</PROTOCOLS>
<OUTPUT scorefxn=“%%energy_function%%NotSymm”/>
</ROSETTASCRIPTS>

### Example 2

### Experimental Methods

### TOXCAT Β-Lactamase Assays

DNA encoding the designs and controls were cloned into the pMAL_dsTβL vector (available at AddGene #73805) using XhoI and SpeI restriction sites and selected by growth on spectinomycin and ampicillin in standard concentrations. For positive controls, the natural ErbB2 and QSOXS2 TM domains were chosen (representing strong and weak homo-oligomers respectively). The monomeric C-terminal portion of human L-selectin (CLS) was chosen as a negative control. Resulting plasmids were transformed into E. coli cloni cells (Lucigen), plated on agar plates containing 50 µl/ml spectinomycin followed by overnight growth in a 37° C. at 200 rpm. Cultures were then inoculated into fresh LB + 50 µl/ml spectinomycin medium to OD₆₀₀ 1 and then plated on petri dishes containing 50 µl/ml spectinomycin, 100 µl/ml ampicillin or 100 µl/ml ampicillin with a range of different chloramphenicol concentrations.

For single-clone growth assays, 2 µl of cultures at OD 0.1 were diluted and plated on square petri dishes containing different chloramphenicol concentrations.

### Deep Sequencing Analysis

A library encoding all of the designed sequences, controls and single-point mutations in defined positions (using NYS codons to encode hydrophobic and small, mildly polar amino acids) was transformed and grown in large 12 cm petri dishes on different chloramphenicol concentrations overnight. Bacteria were harvested and subjected to deep sequencing library preparation and a protocol and analysis as described in Weinstein, J.Y. et al. For viability frequency the WT nucleotide sequences counts were normalized to the total number of counts in each selection. Each frequency was then divided by the reference selection, so that the initial frequency is 1. In a selection regime where there were no WT sequences detected, a pseudo count of 1 was added.

### DNA Sequences of Designs

Coding for proMP 1.1 (SEQ ID No. 1):

CCTTTATCTTTCCTCTTAGGGATACTAGCTGCGCTGGTGGGGTTCATCAT
TGGCTTTTTAGCGGCCTTGATT (SEQ ID No. 15);

Coding for proMP 1.2(trimer; used in proCAR-3; SEQ ID No. 2)

CCTTTGTTATTTATTCTCGTCGCAATACTTGGAGGCTTATTTGGGGCGAT
TGTTGCATTCCTTTTGGCGTTA (SEQ ID No. 16);

Coding for proMP 1.3 (SEQ ID No. 3):

CCGATCCTGTTCGCAATACTGGCGGCTTTCATCGGGGCATTTATAGCTGC
CCTGTTCGTGCTAGTATTGGCA (SEQ ID No. 17);

Coding for proMP 1.4 (SEQ ID No. 4):

CCCTTTGGAGCTTTACTAGCAATCATAGCATTCGTCGTAGGAATGTTATT
CTCAGCATTCGTTTTACTCATC (SEQ ID No. 18);

Coding for proMP 1.5 (SEQ ID No. 5):

CCCTTTAGCTTGTTTTTGGGCGTTATAGCCGGCATTATTGCTGCATTCAT
CGTTTTATTCCTGGCATTACTA (SEQ ID No. 19);

Coding for proMP 1.6 (SEQ ID No. 6):

CCTTTTTTATCGCTTGTTGGTGCGCTAATCGGGGCTTTCATAGCATTTAT
CTTGGCTTTGTTCATTTTGGTT (SEQ ID No. 20);

Coding for proMP 1.7 (SEQ ID No. 7):

CCGATTCTGATCACTTTGGCAATGCTTACGGGAGCAGTGATTGGGGCGAT
CTCGTCTTTTCTCCTAGTGTAT (SEQ ID No. 21);

Coding for proMP 1.8 (SEQ ID No. 8):

CCAGCCTTTTATATTATATTGGCAATTCTCACCTCGTTCATAGCCTATTT
GGTGGGTCTACTCGTGTCTTTT (SEQ ID No. 22);

Coding for proMP 1.9 (SEQ ID No. 9):

CCTATTTACGTTATACTAGCCATCTTGGCCGCGGTATTCACTTGGTTCAT
AGTCCTTATAACTAGCCTGAGT (SEQ ID No. 23);

Coding for proMP 1.10 (SEQ ID No. 10):

CCTACGGTTACGAGTGCGATTCTTGGCGTGTCATTCGGTACCTTTATTAG
CCTCGTAGCTCTGTGGCTTGCA (SEQ ID No. 24);

Coding for proMP 1.11 (SEQ ID No. 11):

CCAGTGATTGCAATCTTAACTTTTATAGTCCTCACTGCGATTTCGGGAGC
GCTGCTCGCTGTTTGGTTCTCC (SEQ ID No. 25);

Coding for proMP 1.12 (SEQ ID No. 12):

CCCATCGTCTTGCTCCTCAGTCTACTCGCCAGTGTATTTGGGGCGTTCAT
CACATTTATTTGGGCTTACTTG (SEQ ID No. 26);

Coding for proMP C1 (monomer; used in proCAR-1):

CTGGTGCTGATTCTGCTGACCTTTGTGCTGTTTGTGTTTATTCTGTATTG
GGTGATTACCTGGTATCTGATT (SEQ ID No. 27);

Coding for proMP C2.1 (dimer; used in proCAR-2; SEQ ID No. 13):

CCGCTGACCGTGGCGCTGATTCTGGGCATCTTCCTGGGCACCTTTATTGC
GTTTTGGGTGGTGTATCTGCTG (SEQ ID No. 28);

Coding for proMP C3.1 (SEQ ID No. 14):

ACCGCGCTGCTGGTGGCGTTTGTGGCGTATTATACCGCGCTGATTGCGCT
GATTTTTGCGATTCTGGCGACC (SEQ ID No. 29);

Coding for proMP C4.1 (tetramer; used in proCAR-4; SEQ ID No. 71):

CCCCTTCTCGTAGCATTGCTGGCCCTTCTGGCCGTCATAGCTGCCCTGCT
GGCCGCAATTTTTGCCCTGCTC (SEQ ID No. 74)

Coding for the monomeric C-terminal portion of human L-selectin (CLS):

CCGCTGTTCATCCCGGTTGCAGTTATGGTTACCGCTTTTAGTGGATTGGG
GTTTATCATCTGGCTGGCTAC (SEQ ID No. 30);

Coding for ErbB2 (erb-b2 receptor tyrosine kinase 2):

TCTATCATCTCTGCGGTGGTTGGCATTCTGCTGGTCGTGGTCTTGGGCGT
GGTCTTTGGCATCCTGAT (SEQ ID No. 31); and

Coding for QSOX2 (quiescin sulfhydryl oxidase 2):

AGCCTATGCGTTGTTTTATACGTGGCATCTAGTTTATTTATGGTCATGTA
CTTCTTC (SEQ ID No. 32).

The sequences below relate to two of the actual TMD sequences used in creating the CAR T cells referred to in the Example section below. SEQ ID No. 35 and SEQ ID No. 36 are similar to SEQ ID No. 2 and SEQ ID No. 13, respectively, except for the first amino acid, which was deleted.

Thus, according to another aspect of the present invention, the designed TMD has the sequence LLFILVAILGGLFGAIVAFLLAL (SEQ ID No. 35), or the sequence LTVALILGIFLGTFIAFWVVYLL (SEQ ID No. 36).

The nucleic acid coding for SEQ ID No. 35:

TTGTTATTTATTCTCGTCGCAATACTTGGAGGCTTATTTGGGGCGATTGT
TGCATTCCTTTTGGCGTTA (SEQ ID No. 41).

The nucleic acid coding for SEQ ID No. 36:

CTGACCGTGGCGCTGATTCTGGGCATCTTCCTGGGCACCTTTATTGCGTT
TTGGGTGGTGTATCTGCTG (SEQ ID No. 42).

A third sequence relates to the actual TMD used in creating proCAR-4 (SEQ ID No. 77) and is missing the first and last amino acid of SEQ ID No. 71. Thus, according to another aspect of the present invention, the designed TMD used in proCAR-4 has the sequence LLVALLALLAVIAALLAAIFAL (SEQ ID No. 72).

The nucleic acid coding for SEQ ID No. 72:

CTTCTCGTAGCATTGCTGGCCCTTCTGGCCGTCATAGCTGCCCTGCTGGC
CGCAATTTTTGCCCTG (SEQ ID No. 75).

The sequences below are the full CAR sequences, introduced into T cells to create the CAR T cells which are clinically relevant, according to some embodiments of the present invention.

Thus, further provided are CAR sequences for the production of CAR T cells as follows:

Coding for ProCAR-2 DNA sequence, based on proMP C2.1 (SEQ ID No. 13):

atgtctagacaggtacaactgcagcagtcaggacctgaactgaagaagcc
tggagagacagtcaagatctcctgcaaggcctctgggtatcctttcacaa
actatggaatgaactgggtgaagcaggctccaggacagggtttaaagtgg
atgggctggattaacacctccactggagagtcaacatttgctgatgactt
caagggacggtttgacttctctttggaaacctctgccaacactgcctatt
tgcagatcaacaacctcaaaagtgaagacatggctacatatttctgtgca
agatgggaggtttaccacggctacgttccttactggggccaagggaccac
ggtcaccgtttcctctggcggtggcggttctggtggcggtggctccggcg
gtggcggttctgacatccagctgacccagtctcacaaattcctgtccact
tcagtaggagacagggtcagcatcacctgcaaggccagtcaggatgtgta
taatgctgttgcctggtatcaacagaaaccaggacaatctcctaaacttc
tgatttactcggcatcctcccggtacactggagtcccttctcgcttcact
ggcagtggctctgggccggatttcactttcaccatcagcagtgtgcaggc
tgaagacctggcagtttatttctgtcagcaacattttcgtactccattca
cgttcggctcggggacaaaattggagatcgaacaaaaactcatctcagaa
gaggatctgaatggggtcaccgtctcttcagcgctgagcaactccatcat
gtacttcagccacttcgtgccggtcttcctgccagcgaagcccaccacga
cgccagcgccgcgaccaccaacaccggcgcccaccatcgcgtcgcagccc
ctgtccctgcgcccagaggcggcccggccagcggcggggggcgcagtgca
cacgagggggctcgatcCCTTTTGGCTGACTGTGGCCCTTATTCTTGGGA TATTCTTAGGAACATTCATAGCATTTTGGGTCGTTTACTTGTTATGGGTG
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCCagagtgaagttcagcaggagcgcagac
gcccccgcgtaccagcagggccagaaccagctctataacgagctcaatct
aggacgaagagaggagtacgatgttttggacaagagacgtggccgggacc
ctgagatggggggaaagccgagaaggaagaaccctcaggaaggcctgtac
aatgaactgcagaaagataagatggcggaggcctacagtgagattgggat
gaaaggcgagcgccggaggggcaaggggcacgatggcctttaccagggtc
tcagtacagccaccaaggacacctacgacgcccttcacatgcaggccctg
ccccctcgctaa (SEQ ID No. 37);

Coding for ProCAR-2 amino acid sequence based on proMP C2.1 (SEQ ID No. 13), wherein small italic letters represent the leader and FRP5 scFv section, the small underscore letter represent the myc tag, the small double underscore letter represent the CD8a stalk (C➔A), the CAPITAL NON-BOLD LETTERS represent the CD28 juxtamembrane and intracellular, the BOLD CAPITAL LETTERS represent the transmembrane sequence derived from proMP C2.1 (SEQ ID No. 36), and the small italic underscore letter represent the CD3 ζ tail:

msrqvqlqqsgpelkkpgetvkisckasgypftnygmnwvkqapgqglkw mgwintstgestfaddfkgrfdfsletsantaylqinnlksedmatyfca rwevyhgyvpywgqgttvtvssggggsggggsggggsdiqltqshkflst svgdrvsitckasqdvynavawyqqkpgqspklliysassrytgvpsrft gsgsgpdftftissvqaedlavyfcqqhfrtpftfgsgtkleieqklise edlngvtvssalsnsimyfshfvpvflpakptttpaprpptpaptiasqp lslrpeaarpaaggavhtrgldPFWLTVALILGIFLGTFIAFWVVYLLWV
RSKRSRLLHSDYMNMTPRRPGPTRKHYQPYAPPRDFAAYRSrvkfsrsad apayqqgqnqlynelnlgrreeydvldkrrgrdpemggkprrknpqegly nelqkdkmaeayseigmkgerrrgkghdglyqglstatkdtydalhmqal
ppr (SEQ ID No. 38);

Coding for ProCAR-3 DNA sequence based on proMP 1.2 (SEQ ID No. 2):

atgtctagacaggtacaactgcagcagtcaggacctgaactgaagaagcc
tggagagacagtcaagatctcctgcaaggcctctgggtatcctttcacaa
actatggaatgaactgggtgaagcaggctccaggacagggtttaaagtgg
atgggctggattaacacctccactggagagtcaacatttgctgatgactt
caagggacggtttgacttctctttggaaacctctgccaacactgcctatt
tgcagatcaacaacctcaaaagtgaagacatggctacatatttctgtgca
agatgggaggtttaccacggctacgttccttactggggccaagggaccac
ggtcaccgtttcctctggcggtggcggttctggtggcggtggctccggcg
gtggcggttctgacatccagctgacccagtctcacaaattcctgtccact
tcagtaggagacagggtcagcatcacctgcaaggccagtcaggatgtgta
taatgctgttgcctggtatcaacagaaaccaggacaatctcctaaacttc
tgatttactcggcatcctcccggtacactggagtcccttctcgcttcact
ggcagtggctctgggccggatttcactttcaccatcagcagtgtgcaggc
tgaagacctggcagtttatttctgtcagcaacattttcgtactccattca
cgttcggctcggggacaaaattggagatcgaacaaaaactcatctcagaa
gaggatctgaatggggtcaccgtctcttcagcgctgagcaactccatcat
gtacttcagccacttcgtgccggtcttcctgccagcgaagcccaccacga
cgccagcgccgcgaccaccaacaccggcgcccaccatcgcgtcgcagccc
ctgtccctgcgcccagaggcggcccggccagcggcggggggcgcagtgca
cacgagggggctcgatCCCTTTTGGCTGTTGTTCATCCTGGTGGCCATTT TGGGTGGACTTTTCGGAGCTATAGTCGCATTTCTCTTGGCCCTATGGGTG
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCCagagtgaagttcagcaggagcgcagac
gcccccgcgtaccagcagggccagaaccagctctataacgagctcaatct
aggacgaagagaggagtacgatgttttggacaagagacgtggccgggacc
ctgagatggggggaaagccgagaaggaagaaccctcaggaaggcctgtac
aatgaactgcagaaagataagatggcggaggcctacagtgagattgggat
gaaaggcgagcgccggaggggcaaggggcacgatggcctttaccagggtc
tcagtacagccaccaaggacacctacgacgcccttcacatgcaggccctg
ccccctcgctaa (SEQ ID No. 39).

ProCAR-3 amino acid sequence based on proMP 1.2 (SEQ ID No. 2), wherein small italic letters represent the leader and FRP5 scFv section, the small underscore letter represent the myc tag, the small double underscore letter represent the CD8a stalk (C➔A), the CAPITAL NON-BOLD LETTERS represent the CD28 juxtamembrane, the BOLD CAPITAL LETTERS represent the transmembrane sequence derived from proMP 1.2 (SEQ ID No. 35), and the small italic underscore letter represent the CD3 ζ tail:

msrqvqlqqsgpelkkpgetvkisckasgypftnygmnwvkqapgqglkw mgwintstgestfaddfkgrfdfsletsantaylqinnlksedmatyfca rwevyhgyvpywgqgttvtvssggggsggggsggggsdiqltqshkflst svgdrvsitckasqdvynavawyqqkpgqspklliysassrytgvpsrft gsgsgpdftftissvqaedlavyfcqqhfrtpftfgsgtkleiegklise edlngvtvssalsnsimyfshfvpvflpakptttpaprpptpaptiasqp lslrpeaarpaaggavhtrgldPFWLLFILVAILGGLFGAIVAFLLALWV
RSKRSRLLHSDYMNMTPRRPGPTRKHYQPYAPPRDFAAYRSrvkfsrsad apayqqgqnqlynelnlgrreeydvldkrrgrdpemggkprrknpqegly nelqkdkmaeayseigmkgerrrgkghdglyqglstatkdtydalhmqal ppr (SEQ ID No. 40).

Coding for ProCAR-4 DNA sequence based on proMP C4.1 (SEQ ID No.71):

atggattttcaggtgcagattttcagcttcctgctaatcagtgcctcagt
cataatgtctagacaggtacaactgcagcagtcaggacctgaactgaaga
agcctggagagacagtcaagatctcctgcaaggcctctgggtatcctttc
acaaactatggaatgaactgggtgaagcaggctccaggacagggtttaaa
gtggatgggctggattaacacctccactggagagtcaacatttgctgatg
acttcaagggacggtttgacttctctttggaaacctctgccaacactgcc
tatttgcagatcaacaacctcaaaagtgaagacatggctacatatttctg
tgcaagatgggaggtttaccacggctacgttccttactggggccaaggga
ccacggtcaccgtttcctctggcggtggcggttctggtggcggtggctcc
ggcggtggcggttctgacatccagctgacccagtctcacaaattcctgtc
cacttcagtaggagacagggtcagcatcacctgcaaggccagtcaggatg
tgtataatgctgttgcctggtatcaacagaaaccaggacaatctcctaaa
cttctgatttactcggcatcctcccggtacactggagtcccttctcgctt
cactggcagtggctctgggccggatttcactttcaccatcagcagtgtgc
aggctgaagacctggcagtttatttctgtcagcaacattttcgtactcca
ttcacgttcggctcggggacaaaattggagatcgaacaaaaactcatctc
agaagaggatctgaatggggtcaccgtctcttcagcgctgagcaactcca
tcatgtacttcagccacttcgtgccggtcttcctgccagcgaagcccacc
acgacgccagcgccgcgaccaccaacaccggcgcccaccatcgcgtcgca
gcccctgtccctgcgcccagaggcggcccggccagcggcggggggcgcag
tgcacacgagggggctcgatcCCTTTTGGCTTCTCGTAGCATTGCTGGCC CTTCTGGCCGTCATAGCTGCCCTGCTGGCCGCAATTTTTGCCCTGTGGGT
GAGGAGTAAGAGGAGCAGGCTCCTGCACAGTGACTACATGAACATGACTC
CCCGCCGCCCCGGGCCCACCCGCAAGCATTACCAGCCCTATGCCCCACCA
CGCGACTTCGCAGCCTATCGCTCCagagtgaagttcagcaggagcgcaga
cgcccccgcgtaccagcagggccagaaccagctctataacgagctcaatc
taggacgaagagaggagtacgatgttttggacaagagacgtggccgggac
cctgagatggggggaaagccgagaaggaagaaccctcaggaaggcctgta
caatgaactgcagaaagataagatggcggaggcctacagtgagattggga
tgaaaggcgagcgccggaggggcaaggggcacgatggcctttaccagggt
ctcagtacagccaccaaggacacctacgacgcccttcacatgcaggccct
gccccctcgctaa(SEQ ID No. 76).

ProCAR-4 amino acid sequence based on proMP C4.1 (SEQ ID No. 71), wherein small italic letters represent the leader and FRP5 scFv section, the small underscore letter represent the myc tag, the small double underscore letter represent the CD8a stalk (C➔A), the CAPITAL NON-BOLD LETTERS represent the CD28 juxtamembrane, the BOLD CAPITAL LETTERS represent the transmembrane sequence derived from proMP C4.1 (SEQ ID No. 72), and the small italic underscore letter represent the CD3 ζ tail:

Mdfqvqifsfllisasvimsrqvqlqqsgpelkkpgetvkisckasgypf tnygmnwvkqapgqglkwmgwintstgestfaddfkgrfdfsletsanta ylqinnlksedmatyfcarwevyhgyvpywgqgttvtvssggggsggggs ggggsdiqltqshkflstsvgdrvsitckasqdvynavawyqqkpgqspk lliysassrytgvpsrftgsgsgpdftftissvqaedlavyfcqqhfrtp ftfgsgtkleieqkliseedlngvtvssalsnsimyfshfvpvflpakpt ttpaprpptpaptiasqplslrpeaarpaaggavhtrgldPFWLLVALLA LLAVIAALLAAIFALWVRSKRSRLLHSDYMNMTPRRPGPTRKHYQPYAPP
RDFAAYRSrvkLsrsadapayqqgqnqlynelnlgrreeydvldkrrgr dpemggkprrknpqeglynelqkdkmaeayseigmkgerrrgkghdglyq glstatkdtydalhmqalppr (SEQ ID No. 77.

### ProMP Peptide Production

Peptides were produced recombinantly as 9His-trpLE fusion proteins in E. coli. To aid purification, analysis and crystallization, all designed sequences were modified to include Glu-Pro-Glu (SEQ ID No. 33) at the amino terminus and Arg-Arg-Leu-Cys (SEQ ID No. 34) at the carboxy terminus based on the favorable properties of the glycophorin A TMD fragment whose structure has been previously determined by x-ray crystallography. Dissolved fusion protein from inclusion bodies was purified on Nickel affinity resin, cyanogen bromide digested and purified by reverse-phase HPLC following the published procedure with the following modifications: the C-terminal Cys sulfhydryl group was protected using 10 mM S-methyl methanethiosulfonate (MMTS, Sigma-Aldrich) during lysis and inclusion body solubilization and peptides were at no time disulfide linked. HPLC-purified peptides were stored as lyophilized products at room temperature until needed.

### SDS-PAGE Analysis

Samples were prepared by drying 15, 45 and 135 µg of each purified peptide taken directly from a HPLC peak or from dry product redissolved in 1,1,1,1,1,1-hexafluoroisopropanol (HFIP, Merck). Samples were lyophilized, re-dissolved in 25 µl 1x NuPAGE LDS sample buffer (Thermo Fisher Scientific) containing 100 mM dithiothreitol and heated for 1 minute at 95° C. Cooled samples were separated on 12% NuPAGE Bis-Tris gels (Thermo Fisher Scientific) at 200 V for 40 minutes and visualized by staining with Coomassie Blue R-250 (Bio-Rad).

### Lipidic Cubic Phase-Induced Membrane Protein Crystallization (LCP)

For reconstitution into LCP, lyophilized peptide was weighed and co-dissolved with appropriate amounts of monoolein (Nu-Chek Prep) in HFIP. Solvent was removed under streaming nitrogen, followed by lyophilization overnight. Peptide-monoolein mix was heated (52° C.) until liquid and mixed 3:2 with 10 mM Tris pH 8.0 for LCP formation using coupled 100 µl gastight Hamilton syringes (Formulatrix) at room temperature. For screening, LCP mixture was dispensed in 100 nl drops onto 96-well glass plates (Molecular Dimensions) with 1000 µl of precipitant solution using a Mosquito LCP robot (TTP Labtech) at room temperature. Plates were sealed and kept at 20° C. in a Rock Imager 1000 (Formulatrix) for incubation and monitoring of crystal formation.

### ProMP Crystallization in Detergent

For reconstitution with detergent, lyophilized peptide was weighed and dissolved in 30 mM detergent (C₈E₄; Anatrace, CsEs; Anatrace) in HFIP. Solvent was removed under streaming nitrogen followed by lyophilization overnight. Peptide-detergent mix was reconstituted in 10 mM Tris pH 8.0. For screening, peptide-detergent mixture was dispensed in 150 nl drops onto SD-2 plates (IDEX Corp) with 150 nl of precipitant solution using a Crystal Phoenix robot (Art Robins Instruments) at room temperature. Droplets were equilibrated against 50 nl of crystallant in the reservoir. Plates were sealed and kept at 20° C. in a Rock Imager 1000 (Formulatrix) for incubation and monitoring of crystal formation.

### Data Collection and Structure Determination

Data were collected on the MX2 beamline of the Australian Synchrotron at a wavelength of 0.9537 Å and a temperature of 100 K. Data were indexed and scaled using XDS and Aimless. Structure factor amplitudes were obtained using cTruncate. 6W9Y was solved with Phaser by molecular replacement using the GpA monomer helix as a search model (PDB code 5EH6). 6W9Z was solved with Phaser by molecular replacement using 5EH6 mutated to the proMP C2.1 (SEQ ID No. 13) sequence in Coot. 6WA0 was solved with Phaser by molecular replacement using the designed trimer as a search model. This resulted in a model that contained good density for two chains, with the final chain of the trimer considerably worse. The third chain was removed and a second molecular replacement job was performed with the first two chains fixed in place and a single helix from the model trimer used as a search model. This resulted in placement of the third helix in an antiparallel direction with respect to the other two chains and this was judged as correct based on comparison of overall R_{free} of each model, average B factors of each chain and visual inspection of the electron density in Coot. Iterative rounds of refinement and model building were performed in PHENIX and Coot.

### ProCAR Construct Preparation

The HER2 specific CAR used has been previously described. Briefly, it contains the FRP5 anti-HER2 scFv, Myc tag, CD8a stalk, CD28 TM/ tail and CD3 ζ tail sequences. PCR primers were used to generate a Cysteine to Alanine mutation in the CD8a stalk region to prevent covalent dimerization. Overlapping PCR used to generate CARs with altered TM domains on the background of the cysteine-mutated CD8a stalk. These constructs were inserted into the pMSCV-IRES-mCherry-II vector via EcoRI/XhoI restriction sites.

### Surface IP and Immunoblot Analysis

2×10⁷ cells per sample were pelleted and washed twice with phosphate-buffered saline (PBS) prior to coating with 20 µg/ml polyclonal anti-mouse IgG for 45 minutes on ice. Cells were washed twice with PBS and lysed in 200 µl PBS/1% IGEPAL-640/P8340 protease inhibitor/10 mM iodoacetamide for 30 mins on ice. Lysate was centrifuged at 20000 g for 10 mins, 10 µl of cleared lysate was taken for 5% input controls with remainder being added to 20 µl Thermofisher Protein G agarose beads and rotated in cold room for 2 hours. Beads were washed with lysis buffer twice then eluted with LDS and boiled. Samples were run on SDS-PAGE and transferred for blotting with 1:2000 anti-Myc primary antibody (Cell signaling #2276) and 1:20000 anti-mouse IgG HRP secondary (Sigma Aldrich A0168).

### Animals

All mice were of an inbred C57B/6J genetic background. All animal experiments were approved and performed in accordance with the regulatory standards of the Walter and Eliza Hall Institute Animal Ethics Committee (Approval: WEHI-2019.020).

### Mouse CD8⁺ T Cell Isolation and Culture

Single-cell suspensions of peripheral lymph nodes from 8-10 week old C57B/6 mice were prepared by mechanically dissociating through a 70 mm cell strainer (BD Biosciences) into cold PBS. CD8 T cells were subsequently selected using the EasySep™ mouse CD8a positive Kit II (Stem Cell Technologies) according to manufacturer’s instructions. Purity was confirmed as more than 95 % using BD LSR II or Cytek Aurora. CD8 T cells were subsequently activated by incubating overnight with Mouse T-Activator CD3/CD28 Dynabeads™ (Gibco) at a bead to cell ratio of 1:1 in mouse T cell medium (mTCM) consisting of Rosewell Park Memorial Institute (RPMI) 1640 Medium (Gibco) supplemented with foetal bovine serum (10%; Bovogen Biological), L-glutamine (2 mM; Gibco), sodium pyruvate (1 mM; Gibco), non-essential amino acids (1x; Sigma-Aldrich), P-mercaptoethanol (50 mM; Sigma-Aldrich) and recombinant human IL-2 (100 IU/ml; PeproTech). Following removal of magnetic beads, T cells were maintained at 1×10⁶ cell/ml in mTCM.

### BW5147 and Primary Mouse CAR-T Cell Generation

Retrovirus for all T cells was produced using calcium phosphate transfection of HEK293T cells. BW5147 cells expressing a destabilized-GFP NFkB reporter element were mixed 1:1 with filtered viral supernatant at a final density of 2.5×10^5 cells/ml. Polybrene transfection reagent (Merck) was added to a final concentration of 8 µg/ml polybrene prior to spinfection (2500 rpm, 37° C., 45 minutes). For primary mouse T cells, plates were coated with 32 µg/ml retronectin (Takara Bio) for 24 hours, before plating of 1×10^6 cells in 1 ml viral supernatant and performing a spinfection (2500 rpm, 37° C., 45 mins). Viral supernatant was removed after 16 hours and replaced with RPMI supplemented with fetal bovine serum (10%; Bovogen Biological) and L-glutamine (2 mM; Gibco) for BW5147 cells, or mTCM for primary T cells.

### CAR-T Cell SKBR3 Co-Culture Assay

5×10⁴ cells/cell-line were aliquoted onto a confluent layer of SKBR3 cells in a 96-well plate at specified time-points. After 8 hrs, all time points were removed from plate and stained with 1:200 anti-CD69 (Biolegend #104524) on ice for 45 minutes. Samples were analyzed on a LSR Fortessa X20 (BD Bioscience) and data were analyzed using FlowJo™ v10 software.

### Flow Cytometry

For CD8 T cell selection and transduction efficiency verification, single cell suspensions were washed and stained with Live/Dead marker Zombie Aqua™ (BioLegend) for 15 minutes at room temperature in PBS, before washing and staining for at least 30 minutes on ice with a panel of monoclonal antibodies (mAbs) including: anti-mouse CD3ε PE (clone 145-2C11, Biolegend), anti-mouse CD8α APC-Cy7 (clone 53-6.7, Biolegend) and anti-mouse Myc-Tag Alexa Fluor® 647 (clone 9B11, Cell Signalling). All samples were analyzed with an LSR II Fortessa (BD Bioscience) or Aurora (Cytek) and data were analyzed using FlowJo™ v10 software.

### Chromium Release Killing Assay

Standard ⁵¹Cr release assays were conducted to assess CAR-T cell cytotoxicity. Target MC57 mouse fibrosarcoma cells stably expressing human HER2 (MC57-HER2) were loaded with 100 mCi ⁵¹Cr for 1 hour at 37° C., washed three times and then 2×10⁴ tumor cells were co-incubated with CAR-T cells at effector-to-target (E:T) ratios ranging from 20:1 to 1.25:1. Supernatants were harvested after 4 hours of co-incubation, plated onto a 96-well scintillator coated LumaPlate (PerkinElmer) and ⁵¹Cr release quantified using a MicroBeta Microplate Counter (PerkinElmer). Target tumor cells incubated in a 5% Triton X-100 solution were used as a maximum release control, while tumor cells incubated in mTCM alone were used as a spontaneous release control. Percent lysis was calculated as: % lysis = ((Experimental release — Spontaneous release) ÷ (Maximum release - Spontaneous release)) 100.

### Incucyte Real-Time Killing Assay

Target MC57 mouse fibrosarcoma cells stably expressing human HER2 (MC57-HER2) were plated at 4×10⁴ cells per well in a 96-well plate with 2×10⁴ CAR-T cells in mTCM containing propidium iodide (PI). The plate was imaged every hour for 24 hours in an Incucyte S3 live cell imager monitoring total PI fluorescence per well (raw counts per µm² per image; units RCU/µm²/image).

### Cytokine Bead Array

To assess cytokine secretion by CAR-T cells, cytokine bead arrays on co-culture supernatants were performed. CAR-T cells (1×10⁵ cells) were washed once in PBS and co-incubated with either mTCM alone, a 1:1 bead to cell ratio of Mouse T-Activator CD3/CD28 Dynabeads™ (Gibco), non-target MC57 parental tumour cells (2×10⁴ cells) or target MC57-HER2 tumour cells (2×10⁴ cells) in triplicate. After 24 hours, supernatants of co-cultures were collected and used in a LEGENDplex Mouse T Helper Cytokine Panel Version 2 Flexi Kit (Biolegend) for IFN-γ, IL-2 and TNFα, and LEGENDplex Mouse Cytokine Panel 2 Flexi Kit (Biolegend) for GM-CSF according to manufacturer’s instructions. All samples were analyzed with an LSR II Fortessa (BD Bioscience) and concentration determined against a standard curve of each analyte using FlowJo™ v10 software.

### In Vivo Tumor Challenge

To measure the ability of proCAR-T cells to control tumor growth in vivo, an aggressive MC38-HER2 cell line was used. Female NOD/SCID/IL2RG^{-/-} (NSG) mice at 6-8 weeks of age (5-6 per group) were given 5×10⁵ MC38-HER2 cells subcutaneously in the flank and treated with 10⁷ CAR⁺CD8⁺ murine T cells 24 hours later. Tumor growth was measured daily using calipers.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An engineered immune cell comprising at least one chimeric antigen receptor (CAR) polypeptide, said CAR polypeptide comprises an endodomain, a transmembrane domain, and an ectodomain, wherein said transmembrane domain comprises or consists of an amino-acid sequence selected from the gr0oup consisting of any one of the sequences corresponding to motifs 1-15, SEQ ID No. 35 and SEQ ID No. 36, and SEQ ID No. 72, wherein:
the sequences corresponding to motif 1 are presented in Table 1;
the sequences corresponding to motif 2 are presented in Table 2;
the sequences corresponding to motif 3 are presented in Table 3;
the sequences corresponding to motif 4 are presented in Table 4;
the sequences corresponding to motif 5 are presented in Table 5;
the sequences corresponding to motif 6 are presented in Table 6;
the sequences corresponding to motif 7 are presented in Table 7;
the sequences corresponding to motif 8 are presented in Table 8;
the sequences corresponding to motif 9 are presented in Table 9;
the sequences corresponding to motif 10 are presented in Table 10;
the sequences corresponding to motif 11 are presented in Table 11;
the sequences corresponding to motif 12 are presented in Table 12;
the sequences corresponding to motif 13 are presented in Table 13;
the sequences corresponding to motif 14 are presented in Table 14; and
the sequences corresponding to motif 15 are presented in Table 15.

2. The engineered immune cell of claim 1, wherein said ectodomain comprises a single-chain variable fragment (scFv).

3. The engineered immune cell of claim 1, wherein said endodomain comprises an intracellular domain selected from the group consisting of CD3 ζ, CD28, 4-1BB, and combinations thereof.

4. The engineered immune cell of claim 1, further comprising a hinge domain between said transmembrane domain and said ectodomain.

5. The engineered immune cell of claim 1, wherein:
said amino-acid sequence corresponding to motif 1 begins at position 7 and ends at position 22, and having a sequence G/A-XXX-A/S-XX-G/A-XXX-G-XXX-A/S;
said amino-acid sequence corresponding to motif 2 begins at position 5 and ends at position 19, and having a sequence I-XX-AI-X-G-G/A/M/S-XX-G-XXX-A/S;
said amino-acid sequence corresponding to motif 3 begins at position 6 and ends at position 16, and having a sequence I-X-A/M/S-A/M/S-XX-G/A-A/S-XX-A/M/S/T;
said amino-acid sequence corresponding to motif 4 begins at position 7 and ends at position 22, and having a sequence G/A-XXX-A-XX-A/M/S-XXX-G-XXX-S/A/F/M/T-XXX-L/I;
said amino-acid sequence corresponding to motif 5 begins at position 6 and ends at position 19, and having a sequence L/I/V-XXXXXXX-A/F/M/S/T-A/F/M/S/T-XXX-L/I/V;
said amino-acid sequence corresponding to motif 6 begins at position 7 and ends at position 23, and having a sequence L/A/M/S-XXX-G/A-XXX-G-A/F/S-XX-A-XXX-A/T/V-XXX-L/F/II-XX-AI-X-G-G/A/M/S-XX-G-XXX-A/S;
said amino-acid sequence corresponding to motif 7 begins at position 4 and ends at position 18, and having a sequence I/L-T-XX-M-X-T-G/A-A/M/S-XX-G-XX-S;
said amino-acid sequence corresponding to motif 8 begins at position 2 and ends at position 20, and having a sequence A-XX-I/L/T/V-X-L/I-X-I/F/L-XXX-FI-A/S-XXX-G/A-L/I;
said amino-acid sequence corresponding to motif 9 begins at position 3 and ends at position 10, and having a sequence A/F/M/S-XX-L/I-XXX-A/S/T;
said amino-acid sequence corresponding to motif 10 begins at position 4 and ends at position 24, and having a sequence T-S/A/F/L/V-XXX-G-XXX-G-XX-I/F/L/V-XXX-A-XXX-A/G/S;
said amino-acid sequence corresponding to motif 11 begins at position 9 and ends at position 20, and having a sequence I/F/L-VL-XXXX-G/A-XXX-A;
said amino-acid sequence corresponding to motif 12 begins at position 10 and ends at position 22, and having a sequence A/F/M/S-XXX-G/A/F/L/M/S-A/F/I/M/S/V-X-I/F/L/M-XXXX-A/T/V;
said amino-acid sequence corresponding to motif 13 begins at position 5 and ends at position 17, and having a sequence A/F/S-L/I-I-X-G-I/A/M/T/V-XX-G-XXX-A/S/V;
said amino-acid sequence corresponding to motif 14 begins at position 2 and ends at position 23, and having a sequence A/G/S-L/I-XX-A-XX-A-XXX-A-L/I/V-X-A/G-L/F-XX-A-XX-A/S;
said amino-acid sequence corresponding to motif 15 begins at position 7 and ends at position 18, and having a sequence L-A/G/S-XX-A/G/S-XXX-A/G/S-XX-A/S;
and “X” is optionally selected from the group of L, I, V, F, M, S, T, W, and Y.

6. The engineered immune cell of claim 1, wherein said CAR polypeptide comprising or consisting of SEQ ID No. 38, SEQ ID No. 40, or SEQ ID No. 77.

7. The engineered immune cell of claim 1, selected from the group consisting of a T cell and a natural killer cell.

8. The engineered immune cell of claim 1, for use in the treatment of cancer and/or adoptive cell therapy.

9. A pharmaceutical composition comprising a therapeutically effective amount of least one engineered immune cell according to claim 1, and a pharmaceutically acceptable carrier.

10. The pharmaceutical composition of claim 9, for use in the treatment of cancer and/or adoptive cell therapy.

11. A method of treating cancer in a subject in need thereof, comprising administering to the subject the pharmaceutical composition of claim 9.

12. The method of claim 11, further comprising, prior to said administering, transducing a plurality of immune cells with a viral vector that comprises a DNA sequence, said DNA sequence encoding said CAR polypeptide.

13. A chimeric antigen receptor (CAR) polypeptide comprising an endodomain, a transmembrane domain, and an ectodomain, wherein said transmembrane domain comprises or consists of an amino-acid sequence selected from the group consisting of any one of the sequences corresponding to motifs 1-15, SEQ ID No. 35 and SEQ ID No. 36, and SEQ ID No. 72, wherein:
the sequences corresponding to motif 1 are presented in Table 1;
the sequences corresponding to motif 2 are presented in Table 2;
the sequences corresponding to motif 3 are presented in Table 3;
the sequences corresponding to motif 4 are presented in Table 4;
the sequences corresponding to motif 5 are presented in Table 5;
the sequences corresponding to motif 6 are presented in Table 6;
the sequences corresponding to motif 7 are presented in Table 7;
the sequences corresponding to motif 8 are presented in Table 8;
the sequences corresponding to motif 9 are presented in Table 9;
the sequences corresponding to motif 10 are presented in Table 10;
the sequences corresponding to motif 11 are presented in Table 11;
the sequences corresponding to motif 12 are presented in Table 12;
the sequences corresponding to motif 13 are presented in Table 13;
the sequences corresponding to motif 14 are presented in Table 14; and
the sequences corresponding to motif 15 are presented in Table 15.

14. The CAR polypeptide of claim 13, further comprising a hinge domain between said transmembrane domain and said ectodomain.

15. A DNA sequence encoding a chimeric antigen receptor (CAR) polypeptide comprising an endodomain, a transmembrane domain, and an ectodomain, wherein said transmembrane domain comprises or consists of an amino-acid sequence selected from the group consisting of any one of the sequences corresponding to motifs 1-15, SEQ ID No. 35 and SEQ ID No. 36, and SEQ ID No. 72, wherein:
the sequences corresponding to motif 1 are presented in Table 1;
the sequences corresponding to motif 2 are presented in Table 2;
the sequences corresponding to motif 3 are presented in Table 3;
the sequences corresponding to motif 4 are presented in Table 4;
the sequences corresponding to motif 5 are presented in Table 5;
the sequences corresponding to motif 6 are presented in Table 6;
the sequences corresponding to motif 7 are presented in Table 7;
the sequences corresponding to motif 8 are presented in Table 8;
the sequences corresponding to motif 9 are presented in Table 9;
the sequences corresponding to motif 10 are presented in Table 10;
the sequences corresponding to motif 11 are presented in Table 11;
the sequences corresponding to motif 12 are presented in Table 12;
the sequences corresponding to motif 13 are presented in Table 13;
the sequences corresponding to motif 14 are presented in Table 14; and
the sequences corresponding to motif 15 are presented in Table 15.

16. The CAR polypeptide of claim 15, further comprising a hinge domain between said transmembrane domain and said ectodomain.

17. The CAR polypeptide of claim 15, comprising or consisting of SEQ ID No. 38, SEQ ID No. 40, or SEQ ID No. 77.

18. A viral vector comprising the DNA sequence of claim 15.

19. A process of obtaining an engineered immune cell, comprising transducing at least one immune cell with the viral vector of claim 18.

20. A designed transmembrane domain comprising or consisting an amino-acid sequence selected from the group consisting of any one of the sequences corresponding to motifs 1-15, SEQ ID No. 35 and SEQ ID No. 36, and SEQ ID No. 72, wherein:
the sequences corresponding to motif 1 are presented in Table 1;
the sequences corresponding to motif 2 are presented in Table 2;
the sequences corresponding to motif 3 are presented in Table 3;
the sequences corresponding to motif 4 are presented in Table 4;
the sequences corresponding to motif 5 are presented in Table 5;
the sequences corresponding to motif 6 are presented in Table 6;
the sequences corresponding to motif 7 are presented in Table 7;
the sequences corresponding to motif 8 are presented in Table 8;
the sequences corresponding to motif 9 are presented in Table 9;
the sequences corresponding to motif 10 are presented in Table 10;
the sequences corresponding to motif 11 are presented in Table 11;
the sequences corresponding to motif 12 are presented in Table 12;
the sequences corresponding to motif 13 are presented in Table 13;
the sequences corresponding to motif 14 are presented in Table 14; and
the sequences corresponding to motif 15 are presented in Table 15.

21. The designed transmembrane domain of claim 20, wherein:
said amino-acid sequence corresponding to motif 1 begins at position 7 and ends at position 22, and having a sequence G/A-XXX-A/S-XX-G/A-XXX-G-XXX-A/S;
said amino-acid sequence corresponding to motif 2 begins at position 5 and ends at position 19, and having a sequence I-XX-AI-X-G-G/A/M/S-XX-G-XXX-A/S;
said amino-acid sequence corresponding to motif 3 begins at position 6 and ends at position 16, and having a sequence I-X-A/M/S-A/M/S-XX-G/A-A/S-XX-A/M/S/T;
said amino-acid sequence corresponding to motif 4 begins at position 7 and ends at position 22, and having a sequence G/A-XXX-A-XX-A/M/S-XXX-G-XXX-S/A/F/M/T-XXX-L/I;
said amino-acid sequence corresponding to motif 5 begins at position 6 and ends at position 19, and having a sequence L/I/V-XXXXXXX-A/F/M/S/T-A/F/M/S/T-XXX-L/I/V;
said amino-acid sequence corresponding to motif 6 begins at position 7 and ends at position 23, and having a sequence L/A/M/S-XXX-G/A-XXX-G-A/F/S-XX-A-XXX-A/T/V-XXX-L/F/II-XX-AI-X-G-G/A/M/S-XX-G-XXX-A/S;
said amino-acid sequence corresponding to motif 7 begins at position 4 and ends at position 18, and having a sequence I/L-T-XX-M-X-T-G/A-A/M/S-XX-G-XX-S;
said amino-acid sequence corresponding to motif 8 begins at position 2 and ends at position 20, and having a sequence A-XX-I/L/T/V-X-L/I-X-I/F/L-XXX-FI-A/S-XXX-G/A-L/I;
said amino-acid sequence corresponding to motif 9 begins at position 3 and ends at position 10, and having a sequence A/F/M/S-XX-L/I-XXX-A/S/T;
said amino-acid sequence corresponding to motif 10 begins at position 4 and ends at position 24, and having a sequence T-S/A/F/L/V-XXX-G-XXX-G-XX-I/F/L/V-XXX-A-XXX-A/G/S;
said amino-acid sequence corresponding to motif 11 begins at position 9 and ends at position 20, and having a sequence I/F/L-VL-XXXX-G/A-XXX-A;
said amino-acid sequence corresponding to motif 12 begins at position 10 and ends at position 22, and having a sequence A/F/M/S-XXX-G/A/F/L/M/S-A/F/I/M/S/V-X-I/F/L/M-XXXX-A/T/V;
said amino-acid sequence corresponding to motif 13 begins at position 5 and ends at position 17, and having a sequence A/F/S-L/I-I-X-G-I/A/M/T/V-XX-G-XXX-A/S/V;
said amino-acid sequence corresponding to motif 14 begins at position 2 and ends at position 23, and having a sequence A/G/S-L/I-XX-A-XX-A-XXX-A-L/I/V-X-A/G-L/F-XX-A-XX-A/S;
said amino-acid sequence corresponding to motif 15 begins at position 7 and ends at position 18, and having a sequence L-A/G/S-XX-A/G/S-XXX-A/G/S-XX-A/S;
and “X” is optionally selected from the group of L, I, V, F, M, S, T, W, and Y.
